SEQUENCE LISTING (61 sequences)

SEQ ID NO: 1 - protein, 448 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 2 - protein, 214 aa, synthetic construct
DIQMTQSPSSLSASVGDRVTITCKASQDVSTAVAWYQQKPGKAPKLLIYSASFRYTGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 359 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 4 - protein, 487 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSGGGGSGGGGSGGSGGEIVLTQSPGTLSLSPGERATLSCRASQSVSSSYLAWYQKKPGQAPRLLIYGASSRATGIPDRFSGSGSGTDFTLTISRLEPEDFAVYYCQQYGSSPLTFGQGTKVEIK

SEQ ID NO: 5 - protein, 344 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 6 - protein, 482 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGSGGEVQLLESGGGLVQPGGSLRLSCAASGFTFSSYAMSWVREAPGKGLEWVSAISGSGGSTYYADSVKGRFTISRDNSKNTLYLQMNSLRAEDTAVYYCAKGSGFDYWGQGTLVTVSSAST

SEQ ID NO: 7 - protein, 362 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 8 - protein, 490 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSGGGGSGGGGSGGGGSGGSGGEIVLTQSPGTLSLSPGERATLSCRASQSVSSSYLAWYQKKPGQAPRLLIYGASSRATGIPDRFSGSGSGTDFTLTISRLEPEDFAVYYCQQYGSSPLTFGQGTKVEIK

SEQ ID NO: 9 - protein, 346 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 10 - protein, 486 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSGGGGSGGGGSGGGGSGGSGGEVQLLESGGGLVQPGGSLRLSCAASGFTFSSYAMSWVREAPGKGLEWVSAISGSGGSTYYADSVKGRFTISRDNSKNTLYLQMNSLRAEDTAVYYCAKGSGFDYWGQGTLVTVSSAST

SEQ ID NO: 11 - protein, 356 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 12 - protein, 484 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSEVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSGGGGSGGGGSGGGGSGGSGGEIVLTQSPGTLSLSPGERATLSCRASQSVSSSYLAWYQKKPGQAPRLLIYGASSRATGIPDRFSGSGSGTDFTLTISRLEPEDFAVYYCQQYGSSPLTFGQGTKVEIK

SEQ ID NO: 13 - protein, 349 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 14 - protein, 487 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGGGGSGGGGSGGGGSGGSGGEVQLLESGGGLVQPGGSLRLSCAASGFTFSSYAMSWVREAPGKGLEWVSAISGSGGSTYYADSVKGRFTISRDNSKNTLYLQMNSLRAEDTAVYYCAKGSGFDYWGQGTLVTVSSAST

SEQ ID NO: 15 - protein, 5 aa, synthetic construct
GYTMN

SEQ ID NO: 16 - protein, 17 aa, synthetic construct
LINPYKGVSTYNQKFKD

SEQ ID NO: 17 - protein, 13 aa, synthetic construct
SGYYGDSDWYFDV

SEQ ID NO: 18 - protein, 122 aa, synthetic construct [sequence omitted]

SEQ ID NO: 19 - protein, 11 aa, synthetic construct
RASQDIRNYLN

SEQ ID NO: 20 - protein, 7 aa, synthetic construct
YTSRLES

SEQ ID NO: 21 - protein, 9 aa, synthetic construct
QQGNTLPWT

SEQ ID NO: 22 - protein, 107 aa, synthetic construct [sequence omitted]

SEQ ID NO: 23 - protein, 5 aa, synthetic construct
SYAMN

SEQ ID NO: 24 - protein, 19 aa, synthetic construct
RIRSKYNNYATYYADSVKG

SEQ ID NO: 25 - protein, 14 aa, synthetic construct
ASNFPASYVSYFAY

SEQ ID NO: 26 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 27 - protein, 14 aa, synthetic construct
GSSTGAVTTSNYAN

SEQ ID NO: 28 - protein, 7 aa, synthetic construct
GTNKRAP

SEQ ID NO: 29 - protein, 9 aa, synthetic construct
ALWYSNLWV

SEQ ID NO: 30 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 31 - protein, 5 aa, synthetic construct
NYYIH

SEQ ID NO: 32 - protein, 17 aa, synthetic construct
WIYPGDGNTKYNEKFKG

SEQ ID NO: 33 - protein, 10 aa, synthetic construct
DSYSNYYFDY

SEQ ID NO: 34 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 35 - protein, 17 aa, synthetic construct
KSSQSLLNSRTRKNYLA

SEQ ID NO: 36 - protein, 7 aa, synthetic construct
WASTRES

SEQ ID NO: 37 - protein, 8 aa, synthetic construct
TQSFILRT

SEQ ID NO: 38 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 39 - protein, 5 aa, synthetic construct
DTYIH

SEQ ID NO: 40 - protein, 17 aa, synthetic construct
RIYPTNGYTRYADSVKG

SEQ ID NO: 41 - protein, 11 aa, synthetic construct
WGGEGFYAMDY

SEQ ID NO: 42 - protein, 120 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGEGFYAMDYWGQGTLVTVSS

SEQ ID NO: 43 - protein, 11 aa, synthetic construct
KASQDVSTAVA

SEQ ID NO: 44 - protein, 7 aa, synthetic construct
SASFRYT

SEQ ID NO: 45 - protein, 9 aa, synthetic construct
QQHYTTPPT

SEQ ID NO: 46 - protein, 107 aa, synthetic construct
DIQMTQSPSSLSASVGDRVTITCKASQDVSTAVAWYQQKPGKAPKLLIYSASFRYTGVPSRFSGSRSGTDFTLTISSLQPEDFATYYCQQHYTTPPTFGQGTKVEIK

SEQ ID NO: 47 - protein, 5 aa, synthetic construct
SYAMS

SEQ ID NO: 48 - protein, 17 aa, synthetic construct
AISGSGGSTYYADSVKG

SEQ ID NO: 49 - protein, 6 aa, synthetic construct
GSGFDY

SEQ ID NO: 50 - protein, 115 aa, synthetic construct
EVQLLESGGGLVQPGGSLRLSCAASGFTFSSYAMSWVREAPGKGLEWVSAISGSGGSTYYADSVKGRFTISRDNSKNTLYLQMNSLRAEDTAVYYCAKGSGFDYWGQGTLVTVSS

SEQ ID NO: 51 - protein, 12 aa, synthetic construct
RASQSVSSSYLA

SEQ ID NO: 52 - protein, 7 aa, synthetic construct
GASSRAT

SEQ ID NO: 53 - protein, 9 aa, synthetic construct
QQYGSSPLT

SEQ ID NO: 54 - protein, 108 aa, synthetic construct
EIVLTQSPGTLSLSPGERATLSCRASQSVSSSYLAWYQKKPGQAPRLLIYGASSRATGIPDRFSGSGSGTDFTLTISRLEPEDFAVYYCQQYGSSPLTFGQGTKVEIK

SEQ ID NO: 55 - protein, 10 aa, synthetic construct
GGGGSGGGGS

SEQ ID NO: 56 - protein, 20 aa, synthetic construct
GGGGSGGGGSGGGGSGGSGG

SEQ ID NO: 57 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 58 - protein, 177 aa, Macaca fascicularis
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 59 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 60 - protein, 14 aa, synthetic construct
HTTFPSSYVSYYGY

SEQ ID NO: 61 - protein, 125 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

(19) United States
(12) Patent Application Publication
(10) Pub. No.: US 2026/0062485 A1
(43) Pub. Date: Mar. 5, 2026

(54) ANTIGEN BINDING MOLECULES

(71) Applicant: Hoffmann-La Roche Inc., Little Falls, NJ (US)

(72) Inventors: Jamal Abu Tarbush, Penzberg (DE); Ali Bransi, Dietikon (CH); John Challier, Zurich (CH); Sofia Helena Linnea Frost, Woodbridge (GB); Guy Georges, Habach (DE); Friederike Hesse, Muenchen (DE); Sabine Imhof-Jung, Krailling (DE); Christian Klein, Bonstetten (CH); Laurent Lariviere, Westendorf (DE)

(73) Assignee: Hoffmann-La Roche Inc., Little Falls, NJ (US)

(21) Appl. No.: 19/208,349

(22) Filed: May 14, 2025

Related U.S. Application Data
(63) Continuation of application No. PCT/EP2023/081550, filed on Nov. 13, 2023.

(30) Foreign Application Priority Data
Nov. 15, 2022 (EP) 22207395.9

(51) Int. Cl. C07K 16/28; A61K 39/00; A61P 35/00

(52) U.S. Cl. CPC C07K 16/2809; A61P 35/00; A61K 2039/505; C07K 2317/52; C07K 2317/565; C07K 2317/71

(57) ABSTRACT

The present invention relates to a pair of binding molecules comprising complementary parts of an effector domain, such binding molecules being capable of forming a functional effector domain when bound to their target antigens on the surface of a cell. Specifically, the invention relates to a pair of binding molecules wherein the complementary parts of the effector domain are complemented with inert complementing domains while the functional effector domain is not formed, providing advantageous properties, such as produceability, stability and/or biological functionality, to the binding molecules.

### CROSS-REFERENCE TO RELATED APPLICATIONS

This application is a continuation of PCT Application No. PCT/EP2023/081550, filed Nov. 13, 2023, which claims priority to European Application No. 22207395.9, filed Nov. 15, 2022, each of which is incorporated by reference in its entirety.

### SEQUENCE LISTING

This application contains a Sequence Listing, which has been submitted electronically in XML format and is hereby incorporated by reference in its entirety. Said XML copy, created on May 12, 2025, is named P37895-US-1-Sequence-Listing.xml and 61,177 bytes in size

### FIELD OF THE INVENTION

The present invention relates to a pair of binding molecules comprising complementary parts of an effector domain, such binding molecules being capable of forming a functional effector domain when bound to their target antigens on the surface of a cell. Specifically, the invention relates to a pair of binding molecules wherein the complementary parts of the effector domain are complemented with inert complementing domains while the functional effector domain is not formed, providing advantageous properties, such as produceability, stability and/or biological functionality, to the binding molecules.

### BACKGROUND

T cell engagers or T cell bispecific antibodies (TCBs) are bispecific antibodies that with one binding moiety recognize a target cell antigen, e.g. a tumor antigen expressed on tumor cells, and with the other binding moiety the T cell receptor. TCBs hold great promise as cancer immunotherapeutics. Crosslinking of CD3 with target cell antigens triggers T cell activation, proliferation and cytokine release, leading to target cell killing (Bacac et al., Clin Cancer Res (2016) 22, 3286-97; Bacac et al., Oncoimmunology (2016) 5, e1203498).

However, due to the potency of T-cell redirection approaches, TCB treatment is sometimes associated with safety liabilities due to on-target off-tumor cytotoxic activity and cytokine release. In particular in the treatment of solid tumors, on-target, off-tumor activity remains a challenge to tackle in order to unleash the full potential of T cell bispecific antibodies.

Clinical experience with T cell bispecific antibodies has so far demonstrated that high rates of durable response are achievable in haematological malignancies, but remains very limited in solid tumors. Various T cell engagers were stopped in phase I trials. Although the reasons for termination of these trials may vary, multiple ones showed adverse events, frequently with CRS despite prophylactic corticosteroid treatment, with limited positive pharmacodynamic signals at rather low doses.

A reason for the limited success of T cell bispecific antibodies particularly in the treatment of solid tumors is the lack of targets in solid tumors that are either tumor specific or only expressed on physiological tissue considered dispensable (such as e.g. CD19 or CD20 on B-cells). Conventional solid tumor T cell bispecific antibodies have so far been hampered by limited efficacy driven by a small therapeutic window due to on-target off-tumor activation of T cells. For example, dose-limiting toxicities have been shown in clinical trials with catumaxomab for the treatment of solid tumors (Mau-Sørensen et al. (2015) Cancer Chemother Pharmacol 75, 1065-1073; Borlak et al. (2016) Oncotarget 7, 28059-28074). Indeed, on-target off-tumor activity of T cell engagers has been a challenge for many years due to the potency of such therapeutic modalities and the lack of truly tumor-specific targets.

Thus, the development of drugs, such as T cell bispecific antibodies, with tumor-restricted activity is required to increase the therapeutic index and improve the outcomes for patients.

Novel approaches are needed to achieve this, and—while technically challenging—generating inactive prodrugs that are mainly or solely active within the tumor microenvironment is a field of intensive research.

To reduce the systemic toxicity often seen with T cell bispecific antibodies, for example Stuhler and colleagues have described a “split” approach for on-target in-situ generation of the CD3 binder generally used in this compound class (Banaszek et al. (2019) Nature Comm 10, 5387; PCT publication no. WO 2013/104804). According to this concept, the functional antibody binding fragment (Fv) of the CD3 binder is split into the VL and the VH domain, which both are not CD3 binding competent as individual domains, and both V domains are comprised separately in separate prodrug molecules, i.e. a CD3-VH prodrug and a CD3-VL prodrug.

VL and VH domains as separate domains are, however, generally less stable than the assembled Fv fragments and often suffer from limited expressibility (Ewert et al. (2003) J Molecular Biol 325, 531-553), as VH domains tend to stabilize VL domains and VL domains may enhance the folding of VH domains. In addition, isolated V domains display hydrophobic surface patches in the interface region to the cognate V domain, and are therefore prone to aggregation as separate domains, especially under stress conditions such as long-term storage or elevated temperature. Many CD3 binders that are frequently used in the industry for T cell bispecific antibodies indeed suffer from these limitations.

Thus, there remains a need for improved “split” approaches that may be applied, for example, to T cell bispecific antibodies.

### SUMMARY OF THE INVENTION

The present invention relates to a pair of binding molecules comprising complementary parts of an effector domain, such binding molecules being capable of forming a functional effector domain when bound to their target antigens on the surface of a cell.

The present inventors have found that complementing the complementary parts of the effector domain with inert complementing domains while the functional effector domain is not formed, provides advantageous properties to the binding molecules, including improved produceability (e.g. increased production yield, easier purification), stability (including after exposure to stress conditions) and/or biological functionality (e.g. reduced assembly in absence of cells expressing the target antigen(s) of the binding molecules).

The complementing domains cover the (potentially hydrophobic) interface between the parts of the effector domain, and assist in folding and stability of the binding molecules. Additionally, the complementing domains can serve as modulators of the association equilibrium of the binding molecules. In non-complemented binding molecules, the equilibrium of assembly depends on the local concentration of the binding molecules and on the association rate constant of the parts of the effector domain. In the complementation approach described herein, the assembly is additionally dependent on the dissociation rate constant of the parts of the effector domain and their respective complementing domains. Thus, with complemented binding molecules, their assembly equilibrium is shifted towards higher concentrations so that target-independent assembly, e.g. in the circulation, is disfavored and target-dependent assembly on target cells is favored. Adjustment of the therapeutic window is possible by fine-tuning the interactions between the parts of the effector domain and the complementing domains.

Accordingly, in a first aspect, the present invention provides a pair of binding molecules, comprising
(a) a first binding molecule comprising (i) a first antigen binding domain capable of binding to a target antigen, (ii) a first part of an effector domain, and (iii) a first complementing domain capable of association with the first part of the effector domain; and
(b) a second binding molecule comprising (i) a second antigen binding domain capable of binding to a target antigen, (ii) a second part of an effector domain, and (iii) a second complementing domain capable of association with the second part of the effector domain;
wherein the first and the second part of the effector domain are capable of associating with each other to form a functional effector domain if the first and the second antigen binding domain bind to their target antigens on the surface of a cell,
wherein the first and the second complementing domain are associated with the first and the second part of the effector domain, respectively, while the first and the second part of the effector domain are not associated with each other.

In another aspect, the invention provides a binding molecule that forms part of the pair of binding molecules of the invention.

According to further aspects of the invention there is provided an isolated polynucleotide encoding the pair of binding molecules or binding molecule of the invention, and a host cell comprising the isolated polynucleotide of the invention. In another aspect is provided a method of producing a (pair of) binding molecule(s), comprising the steps of (a) culturing the host cell of the invention under conditions suitable for the expression of the (pair of) binding molecule(s) and optionally (b) recovering the (pair of) binding molecule(s).

The invention further provides a pharmaceutical composition comprising the pair of binding molecules or binding molecule of the invention and a pharmaceutically acceptable carrier.

Also encompassed by the invention are methods of using the pair of binding molecules, binding molecule and pharmaceutical composition of the invention. In one aspect, the invention provides a pair of binding molecules, binding molecule or pharmaceutical composition according to the invention for use as a medicament. In one aspect is provided a pair of binding molecules, binding molecule, or pharmaceutical composition according to the invention for use in the treatment of a disease. Also provided is the use of pair of binding molecules, binding molecule or pharmaceutical composition according to the invention in the manufacture of a medicament, and the use of a pair of binding molecules, binding molecule or pharmaceutical composition according to the invention in the manufacture of a medicament for the treatment of a disease. The invention also provides a method of treating a disease in an individual, comprising administering to said individual an effective amount of the pair of binding molecules according to the invention.

### DESCRIPTION OF THE INVENTION

### Definitions

Terms are used herein as generally used in the art, unless otherwise defined herein.

As used herein, the terms “first”, “second” or “third” with respect to antigen binding domains etc., are used for convenience of distinguishing when there is more than one of each type of moiety. Use of these terms is not intended to confer a specific order or orientation of the moiety unless explicitly so stated.

The term “binding molecule” as used herein refers to a polypeptide molecule (composed of one or more polypeptide chains) that is capable of binding to an antigen. A binding molecule may be derived from an antibody, and typically comprises an antigen binding domain.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv and scFab), single-domain antibodies, and multispecific antibodies formed from antibody fragments. For a review of certain antibody fragments, see Hollinger and Hudson, Nature Biotechnology 23:1126-1136 (2005).

The terms “full-length antibody,” “intact antibody,” and “whole antibody” are used herein interchangeably to refer to an antibody having a structure substantially similar to a native antibody structure.

An “antigen binding domain” is a molecular domain that is capable of binding to an antigen. The term in particular refers to an antigen binding domain of an antibody, i.e. the part of an antibody that comprises the area which binds to and is complementary to part or all of an antigen. Accordingly, in particular aspects, an antigen binding domain herein is an antigen binding domain of an antibody. Such an antigen binding domain may be provided by, for example, one or more antibody variable domains (also called antibody variable regions). Hence, in particular aspects, an antigen binding domain herein comprises an antibody light chain variable domain (VL) and an antibody heavy chain variable domain (VH). Antigen binding domains may also be provided by non-antibody derived molecules, such as ankyrin repeat protein- or lipocalin-derived binding molecules (DARPins®, Anticalins®).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and complementarity determining regions (CDRs). Sec, e.g., Kindt et al., Kuby Immunology, 6^{th} ed., W.H. Freeman & Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, hinge, CH2 and CH3), which is herein further clarified by referring to “numbering according to Kabat EU index” or “Kabat EU index numbering” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementarity determining regions (CDRs). The FR of a variable domain generally consists of four FR domains: FR1, FR2, FR3, and FR4. Accordingly, the HVR and FR sequences generally appear in the following order in VH (or VL): FR1-HCDR1 (LCDR1)-FR2-HCDR2 (LCDR2)-FR3-HCDR3 (LCDR3)-FR4.

Unless otherwise indicated, CDR residues and other residues in the variable domain (e.g., FR residues) are numbered herein according to Kabat et al., supra.

The term “immunoglobulin molecule” herein refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively. A “Fab molecule” refers to a protein consisting of the VH and CH1 domain of the heavy chain (the “Fab heavy chain”) and the VL and CL domain of the light chain (the “Fab light chain”) of an immunoglobulin.

The term “multispecific” means that a binding molecule (e.g. an antibody) is able to specifically bind to at least two distinct antigenic determinants. A multispecific binding molecule (e.g. antibody) can be, for example, a bispecific binding molecule. Typically, a bispecific binding molecule comprises two antigen binding sites, each of which is specific for a different antigenic determinant. In certain aspects, the multispecific (e.g. bispecific) binding molecule is capable of simultaneously binding two antigenic determinants, particularly two antigenic determinants expressed on the same cell, on neighbouring cells, or cells in the same tissue.

The term “valent” as used herein denotes the presence of a specified number of antigen binding sites in a binding molecule. As such, the term “monovalent binding to an antigen” denotes the presence of one (and not more than one) antigen binding site specific for the antigen in the binding molecule.

An “antigen binding site” refers to the site, i.e. one or more amino acid residues, of a binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigenic determinant” or “antigen” refers to a site (e.g. a contiguous stretch of amino acids or a conformational configuration made up of different regions of non-contiguous amino acids) on a polypeptide macromolecule to which an antigen binding domain binds, forming an antigen binding domain-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). In particular aspects, the antigen is a human protein.

By “T cell antigen” is meant an antigenic determinant expressed on the surface of a T lymphocyte.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In particular aspects, the activating T cell antigen is CD3, particularly the epsilon subunit of CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. Suitable assays to measure T cell activation are known in the art and described herein.

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one aspect, CD3 is human CD3, particularly the epsilon subunit of human CD3 (CD38). The amino acid sequence of human CD38 is shown in SEQ ID NO: 57 (without signal peptide). See also UniProt (www.uniprot.org) entry no. P07766 (version 209), or NCBI (www.ncbi.nlm.nih.gov/) RefSeq NP_000724.1. In other aspects, CD3 is cynomolgus (Macaca fascicularis) CD3, particularly cynomolgus CD38. The amino acid sequence of cynomolgus CD38 is shown in SEQ ID NO: 58 (without signal peptide). See also NCBI GenBank no. BAB71849.1. In certain aspects, the binding molecules of the invention bind to an epitope of CD3 that is conserved among the CD3 antigens from different species, particularly human and cynomolgus CD3. In particular aspects, the binding molecules bind to human CD3.

A “target antigen” as used herein refers to an antigenic determinant presented on the surface of a target cell, for example a cell in a tumor such as a cancer cell or a cell of the tumor stroma (in that case a “tumor antigen”). Preferably, the target antigen is not CD3, and/or is expressed on a different cell than CD3.

“HER2” (also known as erbB-2 or CD340) refers to any native HER2 from any vertebrate source, including mammals such as primates (e.g. humans), non-human primates (e.g. cynomolgus monkeys) and rodents (e.g. mice and rats), unless otherwise indicated. The term encompasses “full-length,” unprocessed HER2 as well as any form of HER2 that results from processing in the cell. The term also encompasses naturally occurring variants of HER2, e.g., splice variants or allelic variants. In one aspect, HER2 is human HER2. The amino acid sequence of human HER2 is shown in UniProt (www.uniprot.org) entry no. Q9UK79 (version 95).

The terms “anti-[protein x] (e.g. CD3) antibody” and “an antibody that binds to [protein x] (e.g. CD3)” refer to an antibody that is capable of binding [protein x] (e.g. CD3) with sufficient affinity such that the antibody is useful as a diagnostic and/or therapeutic agent in targeting [protein x] (e.g. CD3). In one aspect, the extent of binding of an anti-[protein x] (e.g. CD3) antibody to an unrelated, non-[protein x] (e.g. CD3) protein is less than about 10% of the binding of the antibody to [protein x] (e.g. CD3) as measured, e.g., by surface plasmon resonance (SPR). In certain aspects, an antibody that binds to [protein x] (e.g. CD3) has a dissociation constant (K_{D}) of ≤1 μM, ≤500 nM, ≤200 nM, or ≤100 nM. An antibody is said to “specifically bind” to [protein x] (e.g. CD3) when the antibody has a Kp of 1 μM or less, as measured, e.g., by SPR. In certain aspects, an anti-[protein x] (e.g. CD3) antibody binds to an epitope of [protein x] (e.g. CD3) that is conserved among [protein x] (e.g. CD3) from different species.

The term “Fc domain” or “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (Lys447), of the Fc region may or may not be present. Amino acid sequences of heavy chains including an Fc region (or a subunit of an Fc domain as defined herein) are denoted herein without C-terminal glycine-lysine dipeptide if not indicated otherwise. In one aspect, a heavy chain including an Fc region (subunit) as specified herein, comprised in a binding molecule according to the invention, comprises an additional C-terminal glycine-lysine dipeptide (G446 and K447, numbering according to Kabat EU index). In one aspect, a heavy chain including an Fc region (subunit) as specified herein, comprised in a binding molecule according to the invention, comprises an additional C-terminal glycine residue (G446, numbering according to Kabat EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein preferably includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which may be non-identical in the sense that further components fused to each of the subunits (e.g. antigen binding domains) are not the same. In some aspects, the modification promoting the association of the first and the second subunit of the Fc domain comprises an amino acid mutation in the Fc domain, specifically an amino acid substitution. In particular aspects, the modification promoting the association of the first and the second subunit of the Fc domain comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B-cell receptor), and B-cell activation.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example, the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “non-antigen binding”, in relation to a VH and/or VL, is meant that the VH and VL, either alone or in combination, are not capable of specific binding to an antigen, particularly not capable of specific binding to a human antigen. The absence of specific binding of such a VH and/or VL to an antigen (i.e. the absence of any binding that can be discriminated from non-specific interaction) can be determined e.g. by ELISA or surface plasmon resonance.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., an antibody and an antigen). The affinity of a molecule X for its partner Y can generally be represented by the dissociation constant (K_{D}). Affinity can be measured by well-established methods known in the art, including those described herein. A preferred method for measuring affinity is Surface Plasmon Resonance (SPR).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Preferred amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred.

Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, % amino acid sequence identity values are generated using the ggsearch program of the FASTA package version 36.3.8c or later with a BLOSUM50 comparison matrix. The FASTA program package was authored by W. R. Pearson and D. J. Lipman (“Improved Tools for Biological Sequence Analysis”, PNAS 85 (1988) 2444-2448), W. R. Pearson (“Effective protein sequence comparison” Meth. Enzymol. 266 (1996) 227-258), and Pearson et. al. (Genomics 46 (1997) 24-36) and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

By “fused” is meant that the components (e.g. a Fab molecule and an Fc domain subunit) are linked by peptide bonds, either directly or via one or more peptide linkers.

The term “polynucleotide” or “nucleic acid molecule” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a pair of binding molecules or binding molecule of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al. (2017) Nature Medicine 23:815-817, or EP 2 101 823 B1).

An “isolated” nucleic acid molecule refers to a nucleic acid molecule that has been separated from a component of its natural environment. An isolated nucleic acid molecule includes a nucleic acid molecule contained in cells that ordinarily contain the nucleic acid molecule, but the nucleic acid molecule is present extrachromosomally or at a chromosomal location that is different from its natural chromosomal location.

“Isolated polynucleotide (or nucleic acid) encoding a [binding molecule/pair of binding molecules]” refers to one or more polynucleotide molecules encoding the polypeptide chains of the binding molecule(s), e.g. antibody heavy and light chains (or fragments thereof), including such polynucleotide molecule(s) in a single vector or separate vectors, and such polynucleotide molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the binding molecule(s) of the present invention. Host cells include cultured cells, e.g. mammalian cultured cells, such as HEK cells, CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. In one aspect, the host cell of the invention is a eukaryotic cell, particularly a mammalian cell. In one aspect, the host cell is not a cell within a human body.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer. Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

By “[protein x] (e.g. HER2)-positive cancer” or “[protein x] (e.g. HER2)-expressing cancer” is meant a cancer characterized by expression or overexpression of [protein x] (e.g. HER2) in cancer cells. The expression of [protein x] (e.g. HER2) may be determined for example by quantitative real-time PCR (measuring [protein x] (e.g. HER2) mRNA levels), immunohistochemistry (IHC) or western blot assays. In one aspect, the cancer expresses [protein x] (e.g. HER2). In one aspect, the cancer expresses [protein x] (e.g. HER2) in at least 20%, preferably at least 50% or at least 80% of tumor cells as determined by immunohistochemistry (IHC) using an antibody specific for [protein x] (e.g. HER2).

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, the pair of binding molecules of the invention is used to delay development of a disease or to slow the progression of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human.

An “effective amount” of an agent, e.g., a pharmaceutical composition, refers to an amount effective, at dosages and for periods of time necessary, to achieve the desired therapeutic or prophylactic result.

The term “package insert” is used to refer to instructions customarily included in commercial packages of therapeutic products, that contain information about the indications, usage, dosage, administration, combination therapy, contraindications and/or warnings concerning the use of such therapeutic products.

### Pair of Binding Molecules

In a first aspect, the present invention provides a pair of binding molecules, comprising
(a) a first binding molecule comprising (i) a first antigen binding domain capable of binding to a target antigen, (ii) a first part of an effector domain, and (iii) a first complementing domain capable of association with the first part of the effector domain; and
(b) a second binding molecule comprising (i) a second antigen binding domain capable of binding to a target antigen, (ii) a second part of an effector domain, and (iii) a second complementing domain capable of association with the second part of the effector domain;
wherein the first and the second part of the effector domain are capable of associating with each other to form a functional effector domain if the first and the second antigen binding domain bind to their target antigens on the surface of a cell,
wherein the first and the second complementing domain are associated with the first and the second part of the effector domain, respectively, while the first and the second part of the effector domain are not associated with each other.

The pair of binding molecules described above and herein, may incorporate, singly or in combination, any of the features described in the following (unless the context dictates otherwise).

### Effector Domain

According to the invention, each binding molecule of the pair of binding molecules comprises a part of an effector domain, which are capable of forming a functional effector domain if the antigen binding domains of the binding molecules bind to their target antigens on the surface of a cell. The individual parts of the effector domain are not a functional effector domain (i.e. the individual parts of the effector domain do not have the function of the full effector domain).

In some aspects, the effector domain is a dimer.

The functional effector domain can exert a biological function, such as binding to an antigen, activating a cellular signaling pathway, blocking a receptor or the like.

In some aspects, the effector domain is an antigen binding domain. In particular aspects, the effector domain is an anti-CD3 antigen binding domain (i.e. an antigen binding domain capable of binding to CD3).

In some aspects, the functional effector domain is capable of binding to an antigen. In some aspects, the individual parts of the effector domain are not capable of binding to an antigen. In particular aspects, the antigen is a T-cell antigen, particularly an activating T cell antigen. In even more particular aspects, the antigen is CD3, particularly CD38. In some aspects, the antigen is human CD3.

In some aspects, the first part of the effector domain comprises a heavy chain variable region (VH) and the second part of the effector domain comprises a light chain variable region (VL). In some aspects, the first part of the effector domain is a heavy chain variable region (VH) and the second part of the effector domain is a light chain variable region (VL). In some aspects, the first part of the effector domain consists of a heavy chain variable region (VH) and the second part of the effector domain consists of a light chain variable region (VL). In some aspects, the effector domain is an Fv molecule.

In some aspects, the effector domain is a humanized antigen binding domain (i.e. an antigen binding domain of a humanized antibody). In some aspects, the VH and/or the VL of the effector domain is a humanized variable region. In some aspects, the VH and/or the VL of the effector domain comprises an acceptor human framework, e.g. a human immunoglobulin framework or a human consensus framework.

In particular aspects, the VH of the effector domain comprises a heavy chain complementarity determining region (HCDR) 1 of SEQ ID NO: 15, the HCDR 2 of SEQ ID NO: 16 and the HCDR 3 of SEQ ID NO: 17, and the VL of the effector domain comprises a light chain complementarity determining region (LCDR) 1 of SEQ ID NO: 19, a LCDR 2 of SEQ ID NO: 20 and a LCDR 3 of SEQ ID NO: 21. In further aspects, the VH of the effector domain comprises an amino acid sequence that is at least about 95%, 96%, 97%, 98%, 99% or 100% identical to the amino acid sequence of SEQ ID NO: 18, and/or the VL of the effector domain comprises an amino acid sequence that is at least about 95%, 96%, 97%, 98%, 99% or 100% identical to the amino acid sequence of SEQ ID NO: 22.

In some aspects, the VH of the effector domain comprises one or more heavy chain framework sequence (i.e. the FR1, FR2, FR3 and/or FR4 sequence) of the heavy chain variable region sequence of SEQ ID NO: 18. In some aspects, the VH of the effector domain comprises an amino acid sequence that is at least about 95%, 96%, 97%, 98%, or 99% identical to the amino acid sequence of SEQ ID NO: 18. In some aspects, the VH of the effector domain comprises an amino acid sequence that is at least about 95% identical to the amino acid sequence of SEQ ID NO: 18. In some aspects, the VH of the effector domain comprises an amino acid sequence that is at least about 98% identical to the amino acid sequence of SEQ ID NO: 18. In certain aspects, a VH sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 18. In certain aspects, substitutions, insertions, or deletions occur in regions outside the CDRs (i.e., in the FRs). In some aspects, the VH of the effector domain comprises the amino acid sequence of SEQ ID NO: 18. Optionally, the VH of the effector domain comprises the amino acid sequence of SEQ ID NO: 18, including post-translational modifications of that sequence.

In some aspects, the VL of the effector domain comprises one or more light chain framework sequence (i.e. the FR1, FR2, FR3 and/or FR4 sequence) of the light chain variable region sequence of SEQ ID NO: 22. In some aspects, the VL of the effector domain comprises an amino acid sequence that is at least about 95%, 96%, 97%, 98%, or 99% identical to the amino acid sequence of SEQ ID NO: 22. In some aspects, the VL of the effector domain comprises an amino acid sequence that is at least about 95% identical to the amino acid sequence of SEQ ID NO: 22. In some aspects, the VL of the effector domain comprises an amino acid sequence that is at least about 98% identical to the amino acid sequence of SEQ ID NO: 22. In certain aspects, a VL sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 22. In certain aspects, substitutions, insertions, or deletions occur in regions outside the CDRs (i.e., in the FRs). In some aspects, the VL of the effector domain comprises the amino acid sequence of SEQ ID NO: 22. Optionally, the VL of the effector domain comprises the amino acid sequence of SEQ ID NO: 22, including post-translational modifications of that sequence.

In some aspects, the VH of the effector domain comprises an amino acid sequence that is at least about 95%, 96%, 97%, 98%, or 99% identical to the amino acid sequence of SEQ ID NO: 18, and the VL of the effector domain comprises an amino acid sequence that is at least about 95%, 96%, 97%, 98%, or 99% identical to the amino acid sequence of SEQ ID NO: 22. In some aspects, the VH of the effector domain comprises the amino acid sequence of SEQ ID NO: 18 and the VL of the effector domain comprises the amino acid sequence of SEQ ID NO: 22.

In some aspects, the effector domain is an antigen binding domain that binds to CD3 comprising a VH comprising the amino acid sequence of SEQ ID NO: 18 and a VL comprising the amino acid sequence of SEQ ID NO: 22. In some aspects, the effector domain is an antigen binding domain that binds to CD3 comprising a VH sequence of SEQ ID NO: 18 and a VL sequence of SEQ ID NO: 22.

In some aspects, the effector domain is an antigen binding domain that binds to CD3 comprising a VH comprising the heavy chain CDR sequences of the VH of SEQ ID NO: 18, and a VL comprising the light chain CDR sequences of the VL of SEQ ID NO: 22. In some aspects, said antigen binding domain comprises the HCDR1, HCDR2 and HCDR3 amino acid sequences of the VH of SEQ ID NO: 18 and the LCDR1, LCDR2 and LCDR3 amino acid sequences of the VL of SEQ ID NO: 22.

In some aspects, the VH of the effector domain comprises the heavy chain CDR sequences of the VH of SEQ ID NO: 18 and a framework of at least 95%, 96%, 97%, 98% or 99% sequence identity to the framework sequence of the VH of SEQ ID NO: 18. In some aspects, the VH of the effector domain comprises the heavy chain CDR sequences of the VH of SEQ ID NO: 18 and a framework of at least 95% sequence identity to the framework sequence of the VH of SEQ ID NO: 18. In some aspects, the VH of the effector domain comprises the heavy chain CDR sequences of the VH of SEQ ID NO: 18 and a framework of at least 98% sequence identity to the framework sequence of the VH of SEQ ID NO: 18.

In some aspects, the VL of the effector domain comprises the light chain CDR sequences of the VL of SEQ ID NO: 22 and a framework of at least 95%, 96%, 97%, 98% or 99% sequence identity to the framework sequence of the VL of SEQ ID NO: 22. In some aspects, the VL of the effector domain comprises the light chain CDR sequences of the VL of SEQ ID NO: 22 and a framework of at least 95% sequence identity to the framework sequence of the VL of SEQ ID NO: 22. In some aspects, the VL of the effector domain comprises the light chain CDR sequences of the VL of SEQ ID NO: 22 and a framework of at least 98% sequence identity to the framework sequence of the VL of SEQ ID NO: 22.

In some aspects, the effector domain is an antigen binding domain that binds to CD3 comprising a VH sequence as in any of the aspects provided above, and a VL sequence as in any of the aspects provided above.

In further particular aspects, the VH of the effector domain comprises a heavy chain complementarity determining region (HCDR) 1 of SEQ ID NO: 23, the HCDR 2 of SEQ ID NO: 24 and the HCDR 3 of SEQ ID NO: 25, and the VL of the effector domain comprises a light chain complementarity determining region (LCDR) 1 of SEQ ID NO: 27, a LCDR 2 of SEQ ID NO: 28 and a LCDR 3 of SEQ ID NO: 29. In some aspects, the VH of the effector domain comprises an amino acid sequence that is at least about 95%, 96%, 97%, 98%, 99% or 100% identical to the amino acid sequence of SEQ ID NO: 26, and/or the VL of the effector domain comprises an amino acid sequence that is at least about 95%, 96%, 97%, 98%, 99% or 100% identical to the amino acid sequence of SEQ ID NO: 30.

In some aspects, the VH of the effector domain comprises one or more heavy chain framework sequence (i.e. the FR1, FR2, FR3 and/or FR4 sequence) of the heavy chain variable region sequence of SEQ ID NO: 26. In some aspects, the VH of the effector domain comprises an amino acid sequence that is at least about 95%, 96%, 97%, 98%, or 99% identical to the amino acid sequence of SEQ ID NO: 26. In some aspects, the VH of the effector domain comprises an amino acid sequence that is at least about 95% identical to the amino acid sequence of SEQ ID NO: 26. In some aspects, the VH of the effector domain comprises an amino acid sequence that is at least about 98% identical to the amino acid sequence of SEQ ID NO: 26. In certain aspects, a VH sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 26. In certain aspects, substitutions, insertions, or deletions occur in regions outside the CDRs (i.e., in the FRs). In some aspects, the VH of the effector domain comprises the amino acid sequence of SEQ ID NO: 26. Optionally, the VH of the effector domain comprises the amino acid sequence of SEQ ID NO: 26, including post-translational modifications of that sequence.

In some aspects, the VL of the effector domain comprises one or more light chain framework sequence (i.e. the FR1, FR2, FR3 and/or FR4 sequence) of the light chain variable region sequence of SEQ ID NO: 30. In some aspects, the VL of the effector domain comprises an amino acid sequence that is at least about 95%, 96%, 97%, 98%, or 99% identical to the amino acid sequence of SEQ ID NO: 30. In some aspects, the VL of the effector domain comprises an amino acid sequence that is at least about 95% identical to the amino acid sequence of SEQ ID NO: 30. In some aspects, the VL of the effector domain comprises an amino acid sequence that is at least about 98% identical to the amino acid sequence of SEQ ID NO: 30. In certain aspects, a VL sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 30. In certain aspects, substitutions, insertions, or deletions occur in regions outside the CDRs (i.e., in the FRs). In some aspects, the VL of the effector domain comprises the amino acid sequence of SEQ ID NO: 30. Optionally, the VL of the effector domain comprises the amino acid sequence of SEQ ID NO: 30, including post-translational modifications of that sequence.

In some aspects, the VH of the effector domain comprises an amino acid sequence that is at least about 95%, 96%, 97%, 98%, or 99% identical to the amino acid sequence of SEQ ID NO: 26, and the VL of the effector domain comprises an amino acid sequence that is at least about 95%, 96%, 97%, 98%, or 99% identical to the amino acid sequence of SEQ ID NO: 30. In some aspects, the VH of the effector domain comprises the amino acid sequence of SEQ ID NO: 26 and the VL of the effector domain comprises the amino acid sequence of SEQ ID NO: 30.

In some aspects, the effector domain is an antigen binding domain that binds to CD3 comprising a VH comprising the amino acid sequence of SEQ ID NO: 26 and a VL comprising the amino acid sequence of SEQ ID NO: 30. In some aspects, the effector domain is an antigen binding domain that binds to CD3 comprising a VH sequence of SEQ ID NO: 26 and a VL sequence of SEQ ID NO: 30.

In some aspects, the effector domain is an antigen binding domain that binds to CD3 comprising a VH comprising the heavy chain CDR sequences of the VH of SEQ ID NO: 26 and a VL comprising the light chain CDR sequences of the VL of SEQ ID NO: 30. In some aspects, said antigen binding domain comprises the HCDR1, HCDR2 and HCDR3 amino acid sequences of the VH of SEQ ID NO: 26 and the LCDR1, LCDR2 and LCDR3 amino acid sequences of the VL of SEQ ID NO: 30.

In some aspects, the VH of the effector domain comprises the heavy chain CDR sequences of the VH of SEQ ID NO: 26 and a framework of at least 95%, 96%, 97%, 98% or 99% sequence identity to the framework sequence of the VH of SEQ ID NO: 26. In some aspects, the VH of the effector domain comprises the heavy chain CDR sequences of the VH of SEQ ID NO: 26 and a framework of at least 95% sequence identity to the framework sequence of the VH of SEQ ID NO: 26. In some aspects, the VH of the effector domain comprises the heavy chain CDR sequences of the VH of SEQ ID NO: 26 and a framework of at least 98% sequence identity to the framework sequence of the VH of SEQ ID NO: 26.

In some aspects, the VL of the effector domain comprises the light chain CDR sequences of the VL of SEQ ID NO: 30 and a framework of at least 95%, 96%, 97%, 98% or 99% sequence identity to the framework sequence of the VL of SEQ ID NO: 30. In some aspects, the VL of the effector domain comprises the light chain CDR sequences of the VL of SEQ ID NO: 30 and a framework of at least 95% sequence identity to the framework sequence of the VL of SEQ ID NO: 30. In some aspects, the VL of the effector domain comprises the light chain CDR sequences of the VL of SEQ ID NO: 30 and a framework of at least 98% sequence identity to the framework sequence of the VL of SEQ ID NO: 30.

In some aspects, the effector domain is an antigen binding domain that binds to CD3 comprising a VH sequence as in any of the aspects provided above, and a VL sequence as in any of the aspects provided above.

In still other aspects, the VH of the effector domain comprises a heavy chain complementarity determining region (HCDR) 1 of SEQ ID NO: 31, the HCDR 2 of SEQ ID NO: 32 and the HCDR 3 of SEQ ID NO: 33, and the VL of the effector domain comprises a light chain complementarity determining region (LCDR) 1 of SEQ ID NO: 35, a LCDR 2 of SEQ ID NO: 36 and a LCDR 3 of SEQ ID NO: 37. In some aspects, the VH of the effector domain comprises an amino acid sequence that is at least about 95%, 96%, 97%, 98%, 99% or 100% identical to the amino acid sequence of SEQ ID NO: 34, and/or the VL of the effector comprises an amino acid sequence that is at least about 95%, 96%, 97%, 98%, 99% or 100% identical to the amino acid sequence of SEQ ID NO: 38.

In some aspects, the VH of the effector domain comprises one or more heavy chain framework sequence (i.e. the FR1, FR2, FR3 and/or FR4 sequence) of the heavy chain variable region sequence of SEQ ID NO: 34. In some aspects, the VH of the effector domain comprises an amino acid sequence that is at least about 95%, 96%, 97%, 98%, or 99% identical to the amino acid sequence of SEQ ID NO: 34. In some aspects, the VH of the effector domain comprises an amino acid sequence that is at least about 95% identical to the amino acid sequence of SEQ ID NO: 34. In some aspects, the VH of the effector domain comprises an amino acid sequence that is at least about 98% identical to the amino acid sequence of SEQ ID NO: 34. In certain aspects, a VH sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 34. In certain aspects, substitutions, insertions, or deletions occur in regions outside the CDRs (i.e., in the FRs). In some aspects, the VH of the effector domain comprises the amino acid sequence of SEQ ID NO: 34. Optionally, the VH of the effector domain comprises the amino acid sequence of SEQ ID NO: 34, including post-translational modifications of that sequence.

In some aspects, the VL of the effector domain comprises one or more light chain framework sequence (i.e. the FR1, FR2, FR3 and/or FR4 sequence) of the light chain variable region sequence of SEQ ID NO: 38. In some aspects, the VL of the effector domain comprises an amino acid sequence that is at least about 95%, 96%, 97%, 98%, or 99% identical to the amino acid sequence of SEQ ID NO: 38. In some aspects, the VL of the effector domain comprises an amino acid sequence that is at least about 95% identical to the amino acid sequence of SEQ ID NO: 38. In some aspects, the VL of the effector domain comprises an amino acid sequence that is at least about 98% identical to the amino acid sequence of SEQ ID NO: 38. In certain aspects, a VL sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 38. In certain aspects, substitutions, insertions, or deletions occur in regions outside the CDRs (i.e., in the FRs). In some aspects, the VL of the effector domain comprises the amino acid sequence of SEQ ID NO: 38. Optionally, the VL of the effector domain comprises the amino acid sequence of SEQ ID NO: 38, including post-translational modifications of that sequence.

In some aspects, the VH of the effector domain comprises an amino acid sequence that is at least about 95%, 96%, 97%, 98%, or 99% identical to the amino acid sequence of SEQ ID NO: 34, and the VL of the effector domain comprises an amino acid sequence that is at least about 95%, 96%, 97%, 98%, or 99% identical to the amino acid sequence of SEQ ID NO: 38. In some aspects, the VH of the effector domain comprises the amino acid sequence of SEQ ID NO: 34 and the VL of the effector domain comprises the amino acid sequence of SEQ ID NO: 38.

In some aspects, the effector domain is an antigen binding domain that binds to CD3 comprising a VH comprising the amino acid sequence of SEQ ID NO: 34 and a VL comprising the amino acid sequence of SEQ ID NO: 38. In some aspects, the effector domain is an antigen binding domain that binds to CD3 comprising a VH sequence of SEQ ID NO: 34 and a VL sequence of SEQ ID NO: 38.

In some aspects, the effector domain is an antigen binding domain that binds to CD3 comprising a VH comprising the heavy chain CDR sequences of the VH of SEQ ID NO: 34, and a VL comprising the light chain CDR sequences of the VL of SEQ ID NO: 38. In some aspects, said antigen binding domain comprises the HCDR1, HCDR2 and HCDR3 amino acid sequences of the VH of SEQ ID NO: 34 and the LCDR1, LCDR2 and LCDR3 amino acid sequences of the VL of SEQ ID NO: 38.

In some aspects, the VH of the effector domain comprises the heavy chain CDR sequences of the VH of SEQ ID NO: 34 and a framework of at least 95%, 96%, 97%, 98% or 99% sequence identity to the framework sequence of the VH of SEQ ID NO: 34. In some aspects, the VH of the effector domain comprises the heavy chain CDR sequences of the VH of SEQ ID NO: 34 and a framework of at least 95% sequence identity to the framework sequence of the VH of SEQ ID NO: 34. In some aspects, the VH of the effector domain comprises the heavy chain CDR sequences of the VH of SEQ ID NO: 34 and a framework of at least 98% sequence identity to the framework sequence of the VH of SEQ ID NO: 34.

In some aspects, the VL of the effector domain comprises the light chain CDR sequences of the VL of SEQ ID NO: 38 and a framework of at least 95%, 96%, 97%, 98% or 99% sequence identity to the framework sequence of the VL of SEQ ID NO: 38. In some aspects, the VL of the effector domain comprises the light chain CDR sequences of the VL of SEQ ID NO: 38 and a framework of at least 95% sequence identity to the framework sequence of the VL of SEQ ID NO: 38. In some aspects, the VL of the effector domain comprises the light chain CDR sequences of the VL of SEQ ID NO: 38 and a framework of at least 98% sequence identity to the framework sequence of the VL of SEQ ID NO: 38.

In some aspects, the effector domain is an antigen binding domain, particularly an Fv molecule, that binds to CD3 comprising a VH sequence as in any of the aspects provided above, and a VL sequence as in any of the aspects provided above.

In other aspects, the VH of the effector domain comprises a heavy chain complementarity determining region (HCDR) 1 of SEQ ID NO: 23, the HCDR 2 of SEQ ID NO: 24 and the HCDR 3 of SEQ ID NO: 60, and the VL of the effector domain comprises a light chain complementarity determining region (LCDR) 1 of SEQ ID NO: 27, a LCDR 2 of SEQ ID NO: 28 and a LCDR 3 of SEQ ID NO: 29. In some aspects, the VH of the effector domain comprises an amino acid sequence that is at least about 95%, 96%, 97%, 98%, 99% or 100% identical to the amino acid sequence of SEQ ID NO: 61, and/or the VL of the effector domain comprises an amino acid sequence that is at least about 95%, 96%, 97%, 98%, 99% or 100% identical to the amino acid sequence of SEQ ID NO: 30.

In some aspects, the VH of the effector domain comprises one or more heavy chain framework sequence (i.e. the FR1, FR2, FR3 and/or FR4 sequence) of the heavy chain variable region sequence of SEQ ID NO: 61. In some aspects, the VH of the effector domain comprises an amino acid sequence that is at least about 95%, 96%, 97%, 98%, or 99% identical to the amino acid sequence of SEQ ID NO: 61. In some aspects, the VH of the effector domain comprises an amino acid sequence that is at least about 95% identical to the amino acid sequence of SEQ ID NO: 61. In some aspects, the VH of the effector domain comprises an amino acid sequence that is at least about 98% identical to the amino acid sequence of SEQ ID NO: 61. In certain aspects, a VH sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 61. In certain aspects, substitutions, insertions, or deletions occur in regions outside the CDRs (i.e., in the FRs). In some aspects, the VH of the effector domain comprises the amino acid sequence of SEQ ID NO: 61. Optionally, the VH of the effector domain comprises the amino acid sequence of SEQ ID NO: 61, including post-translational modifications of that sequence.

In some aspects, the VL of the effector domain comprises one or more light chain framework sequence (i.e. the FR1, FR2, FR3 and/or FR4 sequence) of the light chain variable region sequence of SEQ ID NO: 30. In some aspects, the VL of the effector domain comprises an amino acid sequence that is at least about 95%, 96%, 97%, 98%, or 99% identical to the amino acid sequence of SEQ ID NO: 30. In some aspects, the VL of the effector domain comprises an amino acid sequence that is at least about 95% identical to the amino acid sequence of SEQ ID NO: 30. In some aspects, the VL of the effector domain comprises an amino acid sequence that is at least about 98% identical to the amino acid sequence of SEQ ID NO: 30. In certain aspects, a VL sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to CD3. In certain aspects, a total of 1 to 10 amino acids have been substituted, inserted and/or deleted in the amino acid sequence of SEQ ID NO: 30. In certain aspects, substitutions, insertions, or deletions occur in regions outside the CDRs (i.e., in the FRs). In some aspects, the VL of the effector domain comprises the amino acid sequence of SEQ ID NO: 30. Optionally, the VL of the effector domain comprises the amino acid sequence of SEQ ID NO: 30, including post-translational modifications of that sequence.

In some aspects, the VH of the effector domain comprises an amino acid sequence that is at least about 95%, 96%, 97%, 98%, or 99% identical to the amino acid sequence of SEQ ID NO: 61, and the VL of the effector domain comprises an amino acid sequence that is at least about 95%, 96%, 97%, 98%, or 99% identical to the amino acid sequence of SEQ ID NO: 30. In some aspects, the VH of the effector domain comprises the amino acid sequence of SEQ ID NO: 61 and the VL of the effector domain comprises the amino acid sequence of SEQ ID NO: 30.

In some aspects, the effector domain is an antigen binding domain that binds to CD3 comprising a VH comprising the amino acid sequence of SEQ ID NO: 61 and a VL comprising the amino acid sequence of SEQ ID NO: 30. In some aspects, the effector domain is an antigen binding domain that binds to CD3 comprising a VH sequence of SEQ ID NO: 61 and a VL sequence of SEQ ID NO: 30.

In some aspects, the effector domain is an antigen binding domain that binds to CD3 comprising a VH comprising the heavy chain CDR sequences of the VH of SEQ ID NO: 61 and a VL comprising the light chain CDR sequences of the VL of SEQ ID NO: 30. In some aspects, said antigen binding domain comprises the HCDR1, HCDR2 and HCDR3 amino acid sequences of the VH of SEQ ID NO: 61 and the LCDR1, LCDR2 and LCDR3 amino acid sequences of the VL of SEQ ID NO: 30.

In some aspects, the VH of the effector domain comprises the heavy chain CDR sequences of the VH of SEQ ID NO: 61 and a framework of at least 95%, 96%, 97%, 98% or 99% sequence identity to the framework sequence of the VH of SEQ ID NO: 61. In some aspects, the VH of the effector domain comprises the heavy chain CDR sequences of the VH of SEQ ID NO: 61 and a framework of at least 95% sequence identity to the framework sequence of the VH of SEQ ID NO: 61. In some aspects, the VH of the effector domain comprises the heavy chain CDR sequences of the VH of SEQ ID NO: 61 and a framework of at least 98% sequence identity to the framework sequence of the VH of SEQ ID NO: 61.

In some aspects, the VL of the effector domain comprises the light chain CDR sequences of the VL of SEQ ID NO: 30 and a framework of at least 95%, 96%, 97%, 98% or 99% sequence identity to the framework sequence of the VL of SEQ ID NO: 30. In some aspects, the VL of the effector domain comprises the light chain CDR sequences of the VL of SEQ ID NO: 30 and a framework of at least 95% sequence identity to the framework sequence of the VL of SEQ ID NO: 30. In some aspects, the VL of the effector domain comprises the light chain CDR sequences of the VL of SEQ ID NO: 30 and a framework of at least 98% sequence identity to the framework sequence of the VL of SEQ ID NO: 30.

In some aspects, the effector domain is an antigen binding domain that binds to CD3 comprising a VH sequence as in any of the aspects provided above, and a VL sequence as in any of the aspects provided above.

### Complementing Domain

According to the invention, each binding molecule of the pair of binding molecules comprises a complementing domain, which associates with the part of an effector domain in such binding molecule while the parts of the effector domain are not associated with each other.

The complementing domains are complementary to the respective parts of the effector domains, but do not form a functional effector domain with the respective part of the effector domain.

In some aspects, the first and the second complementing domain are not capable of forming a functional effector domain with a part of the effector domain. In some aspects, the first and the second complementing domain are not forming a functional effector domain when associated with a part of the effector domain. In some aspects, the first part of the effector domain and the first complementing domain and/or the second part of the effector domain and the second complementing domain are not forming a functional effector domain when associated with each other.

The complementing domains may also be complementary to each other.

In some aspects, the first and the second complementing domain are capable of associating with each other. In some aspects, the first and second complementing domain associate with each other if the first and the second part of the effector domain are associated with each other.

In some aspects, the first complementing domain comprises a VL and the second complementing domain comprises a VH. In some aspects, the first complementing domain is a VL and the second complementing domain is a VH. In some aspects, the first complementing domain consists of a VL and the second complementing domain consists of a VH.

In some aspects, the VH and the VL of the complementing domains are non-antigen binding (either alone (i.e. not associated to one another) or in combination (i.e. associated to one another, as in an antigen binding domain)).

In some aspects, the VH of the second complementing domain comprises a heavy chain complementarity determining region (HCDR) 1 of SEQ ID NO: 47, the HCDR 2 of SEQ ID NO: 48 and the HCDR 3 of SEQ ID NO: 49, and the VL of the first complementing domain comprises a light chain complementarity determining region (LCDR) 1 of SEQ ID NO: 51, a LCDR 2 of SEQ ID NO: 52 and a LCDR 3 of SEQ ID NO: 53. In some aspects, the VH of the second complementing domain comprises an amino acid sequence that is at least about 95%, 96%, 97%, 98%, 99% or 100% identical to the amino acid sequence of SEQ ID NO: 50, and/or the VL of the first complementing domain comprises an amino acid sequence that is at least about 95%, 96%, 97%, 98%, 99% or 100% identical to the amino acid sequence of SEQ ID NO: 54.

In some aspects, the VH of the second complementing domain comprises one or more heavy chain framework sequence (i.e. the FR1, FR2, FR3 and/or FR4 sequence) of the heavy chain variable region sequence of SEQ ID NO: 50. In some aspects, the VH of the second complementing domain comprises an amino acid sequence that is at least about 95%, 96%, 97%, 98%, or 99% identical to the amino acid sequence of SEQ ID NO: 50. In some aspects, the VH of the second complementing domain comprises an amino acid sequence that is at least about 95% identical to the amino acid sequence of SEQ ID NO: 50. In some aspects, the VH of the second complementing domain comprises an amino acid sequence that is at least about 98% identical to the amino acid sequence of SEQ ID NO: 50. In some aspects, the VH of the second complementing domain comprises the amino acid sequence of SEQ ID NO: 50. Optionally, the VH of the second complementing domain comprises the amino acid sequence of SEQ ID NO: 50, including post-translational modifications of that sequence.

In some aspects, the VL of the first complementing domain comprises one or more light chain framework sequence (i.e. the FR1, FR2, FR3 and/or FR4 sequence) of the light chain variable region sequence of SEQ ID NO: 54. In some aspects, the VL of the first complementing domain comprises an amino acid sequence that is at least about 95%, 96%, 97%, 98%, or 99% identical to the amino acid sequence of SEQ ID NO: 54. In some aspects, the VL of the first complementing domain comprises an amino acid sequence that is at least about 95% identical to the amino acid sequence of SEQ ID NO: 54. In some aspects, the VL of the first complementing domain comprises an amino acid sequence that is at least about 98% identical to the amino acid sequence of SEQ ID NO: 54. In some aspects, the VL of the first complementing domain comprises the amino acid sequence of SEQ ID NO: 54. Optionally, the VL of the first complementing domain comprises the amino acid sequence of SEQ ID NO: 54, including post-translational modifications of that sequence.

In some aspects, the VH of the second complementing domain comprises an amino acid sequence that is at least about 95%, 96%, 97%, 98%, or 99% identical to the amino acid sequence of SEQ ID NO: 50, and the VL of the first complementing domain comprises an amino acid sequence that is at least about 95%, 96%, 97%, 98%, or 99% identical to the amino acid sequence of SEQ ID NO: 54. In some aspects, the VH of the second complementing domain comprises the amino acid sequence of SEQ ID NO: 50 and the VL of the first complementing domain comprises the amino acid sequence of SEQ ID NO: 54.

In some aspects, the first and second complementing domain are part of a non-antigen binding domain comprising a VH comprising the amino acid sequence of SEQ ID NO: 50 and a VL comprising the amino acid sequence of SEQ ID NO: 54. In some aspects, the first and the second complementing domain are part of a non-antigen binding domain comprising a VH sequence of SEQ ID NO: 50 and a VL sequence of SEQ ID NO: 54.

In some aspects, the first and the second complementing domain are part of a non-antigen binding domain comprising a VH comprising the heavy chain CDR sequences of the VH of SEQ ID NO: 50, and a VL comprising the light chain CDR sequences of the VL of SEQ ID NO: 54. In some aspects, said non-antigen binding domain comprises the HCDR1, HCDR2 and HCDR3 amino acid sequences of the VH of SEQ ID NO: 50 and the LCDR1, LCDR2 and LCDR3 amino acid sequences of the VL of SEQ ID NO: 54.

In some aspects, the VH of the second complementing domain comprises the heavy chain CDR sequences of the VH of SEQ ID NO: 50 and a framework of at least 95%, 96%, 97%, 98% or 99% sequence identity to the framework sequence of the VH of SEQ ID NO: 50. In some aspects, the VH of the second complementing domain comprises the heavy chain CDR sequences of the VH of SEQ ID NO: 50 and a framework of at least 95% sequence identity to the framework sequence of the VH of SEQ ID NO: 50. In some aspects, the VH of the second complementing domain comprises the heavy chain CDR sequences of the VH of SEQ ID NO: 50 and a framework of at least 98% sequence identity to the framework sequence of the VH of SEQ ID NO: 50.

In some aspects, the VL of the first complementing domain comprises the light chain CDR sequences of the VL of SEQ ID NO: 54 and a framework of at least 95%, 96%, 97%, 98% or 99% sequence identity to the framework sequence of the VL of SEQ ID NO: 54. In some aspects, the VL of the first complementing domain comprises the light chain CDR sequences of the VL of SEQ ID NO: 54 and a framework of at least 95% sequence identity to the framework sequence of the VL of SEQ ID NO: 54. In some aspects, the VL of the first complementing domain comprises the light chain CDR sequences of the VL of SEQ ID NO: 54 and a framework of at least 98% sequence identity to the framework sequence of the VL of SEQ ID NO: 54.

In some aspects, the first and the second complementing domain are part of a non-antigen binding domain comprising a VH sequence as in any of the aspects provided above, and a VL sequence as in any of the aspects provided above.

In some aspects, the first part of the effector domain comprises a first VH and the first complementing domain comprises a first VL, and the second part of the effector domain comprises a second VL and the second complementing domain comprises a second VH. In some aspects, the first part of the effector domain is a first VH and the first complementing domain is a first VL, and the second part of the effector domain is a second VL and the second complementing domain is a second VH. In some aspects, the first part of the effector domain consists of a first VH and the first complementing domain consists of a first VL, and the second part of the effector domain consists of a second VL and the second complementing domain consists of a second VH.

In some aspects, the first part of the effector domain and the first complementing domain, and/or the second part of the effector domain and the second complementing domain are fused to each other. In some aspects, the first part of the effector domain is fused at its C-terminus to the N-terminus of the first complementing domain, and/or the second part of the effector domain is fused at its C-terminus to the N-terminus of the second complementing domain.

### Charged Residues

The complementing domains and/or the parts of the effector domain may comprise amino acid substitutions, specifically substitutions by charged amino acid residues, which modulate the affinity between the complementing domains and/or the parts of the effector domain. For example, introduction of amino acid residues of opposite charge in the two complementing domains will increase the affinity of the two complementing domains to each other (and hence increase the strength and propensity of association of the two complementing domains with each other).

Conversely, introduction of amino acid residues of same charge in the two complementing domains will decrease the affinity of the two complementing domains to each other (and hence decrease the strength and propensity of association of the two complementing domains with each other). The same may be applied mutatis mutandis to the two parts of the effector domain.

In some aspects, each of the second VH and the first VL and/or each of the first VH and the second VL according to the above aspects, particularly each of the second VH and the first VL, comprise an amino acid substitution wherein an amino acid residue is substituted for a charged replacement amino acid residue, wherein (i) the replacement amino acid residues in the VH and the VL are of opposite charge, or (ii) the replacement amino acid residues in the VH and the VL are of the same charge.

In another aspect, each of first VH and the first VL and/or each of the second VH and the second VL comprise an amino acid substitution, wherein an amino acid residue is substituted for a charged replacement amino acid residue, wherein (i) the replacement amino acid residues in the VH and the VL are of opposite charge, or (ii) the replacement amino acid residues in the VH and the VL are of the same charge.

In some aspects, (i) the replacement amino acid residue in the VH is a positively charged amino acid residue and the replacement amino acid residue in the VL is a negatively charged amino acid residue, or the replacement amino acid residue in the VH is a negatively charged amino acid residue and the replacement amino acid residue in the VL is a positively charged amino acid residue, or (ii) the replacement amino acid residues in the VH and the VL are each a positively charged amino acid residue, or the replacement amino acid residues in the VH and the VL are each a negatively charged amino acid residue. In some aspects, the positively charged amino acid residue is lysine (K), arginine (R) or histidine (H), particularly lysine (K) or arginine (R), most particularly lysine (K). In some aspects, the negatively charged amino acid residue is glutamic acid (E) or aspartic acid (D), particularly glutamic acid (E).

In some aspects, the amino acid substitution is in the framework region of the VH and VL.

In some aspects, the amino acid substitution is at a position at the interface between the VH and VL (when associated to each other).

In some aspects, the amino acid substitution is at position 39 of the VH and at position 38 of the VL (numbering according to Kabat EU index).

In some aspects, the amino acid substitution in the VH is Q39K or Q39E, and/or the amino acid substitution in the VL is Q38K or Q38E (numbering according to Kabat EU index).

In some aspects, the amino acid substitution in the VH is Q39K and the amino acid substitution in the VL is Q38E, the amino acid substitution in the VH is Q39E and the amino acid substitution in the VL is Q38K, the amino acid substitution in the VH is Q39K and the amino acid substitution in the VL is Q38K, or the amino acid substitution in the VH is Q39E and the amino acid substitution in the VL is Q38E.

Suitable amino acid substitutions are also described e.g. in Igawa et al (Prot Eng Des Sel (2010) 23, 667-677) or European patent application EP1870459 (A1) (incorporated herein by reference in their entirety).

### Antigen Binding Domain

According to the invention, each binding molecule of the pair of binding molecules comprises an antigen binding domain, which antigen binding domains are capable of binding to one or more target antigen. Binding of the binding molecules to their target antigen(s) on the surface of a cell through their antigen binding domains allows the two parts of the effector domain to associate with each other and form the functional effector domain.

Without wishing to be bound by theory, if the two parts of the effector domain are in sufficient proximity to each other (through the binding of the binding molecules to their target antigen(s) on the surface of a cell), they will dissociate from their respective complementing domains and instead associate with each other to form the functional effector domain.

Binding of each binding molecule may be monovalent (i.e. a binding molecule comprising only a single antigen binding domain) or multivalent, e.g. bivalent (i.e. a binding molecule comprising more than one, e.g. two, antigen binding domains).

Each of the binding molecules may be monospecific (i.e. all binding domains of a binding molecule binding to the same target antigen) or multispecific, e.g. bispecific (i.e at least one antigen binding domain of a binding molecule binding to one target antigen and at least one antigen binding domain of the binding molecule binding to a different target antigen).

Furthermore, binding molecules comprised in the pair of binding molecules according to the invention may have the same or different binding specificity (i.e. both binding molecules bind to the same target antigen(s) or the two binding molecules bind to different target antigen(s)).

In some aspects, the first binding molecule comprises a third antigen binding domain capable of binding to a target antigen, and/or the second binding molecule comprises a fourth antigen binding domain capable of binding to a target antigen.

In some aspects, each binding molecule comprises a single antigen binding domain capable of binding to a target antigen. In other aspects, each binding molecule comprises two antigen binding domains capable of binding to a target antigen.

In some aspects, the first, the second, the third (where present) and/or the fourth (where present) antigen binding domain is an antigen binding domain selected from the group consisting of an Fv molecule, a scFv molecule, a Fab molecule and a single-domain antibody.

In some aspects, the first, the second, the third (where present) and/or the fourth (where present) antigen binding domain is a Fab molecule. In some aspects, the first, the second, the third (where present) and the fourth (where present) antigen binding domain are each a Fab molecule.

In some aspects, each binding molecule comprises a single antigen binding domain capable of binding to a target antigen, wherein said antigen binding domain is a Fab molecule. In other aspects, each binding molecules comprises two antigen binding domains capable of binding to a target antigen, wherein said antigen binding domains are Fab molecules.

In some aspects, the first and the second antigen binding domain bind to the same target antigen. In some aspects, the first, the second, the third (where present) and the fourth (where present) antigen binding domain bind to the same target antigen.

In some aspects, the first and the second antigen binding domain bind to different target antigens. In some aspects, the first, the second, the third (where present) and the fourth (where present) antigen binding domain bind to different target antigens.

In some aspects, the first and the second antigen binding domain bind to the same target antigen, and the third (where present) and the fourth (where present) antigen binding domain bind to the same target antigen, wherein the target antigen bound by the first and the second antigen binding domain is different from the target antigen bound by the third (where present) and the fourth (where present) antigen binding domain.

In particular aspects, the first and the third (where present) antigen binding domain bind to the same target antigen, and the second and the fourth (where present) antigen binding domain bind to the same target antigen, wherein the target antigen bound by the first and the third (where present) antigen binding domain is different from the target antigen bound by the second and the fourth (where present) antigen binding domain.

In some aspects, the effector domain is an antigen binding domain, and the target antigen of the first, the second, the third (where present) and the fourth (where present) antigen binding domain is different from the antigen bound by the effector domain (i.e. the antigen that the functional effector domain is capable of binding, e.g. CD3). In some aspects, the effector domain is an antigen binding domain, and none of the antigen binding domains of the first and the second binding molecule is capable of binding the antigen bound by the effector domain (i.e. the antigen that the functional effector domain is capable of binding, e.g. CD3).

In some aspects, the target antigen of the first, the second, the third (where present) and the fourth (where present) antigen binding domain is not CD3, particularly human CD3. In some aspects, none of the antigen binding domains of the first and the second binding molecule is capable of binding to CD3, particularly human CD3.

In some aspects, the target antigens of the first, the second, the third (where present) and fourth (where present) antigen binding domain are expressed on the same cell, on neighbouring cells, or cells in the same tissue (i.e. cells that are, if not the same, in close proximity to each other). In some aspects, the target antigens of the first, the second, the third (where present) and fourth (where present) antigen binding domain are expressed on the same cell.

In some aspects, the cell is a tumor cell. (e.g. a cancer cell or a cell of the tumor stroma, particularly a cancer cell). In some aspects, the target antigen of the first, the second, the third (where present) and the fourth (where present) antigen binding domain is a tumor antigen.

An exemplary target antigen is HER2.

In some aspects, the target antigen of the first, the second, the third (where present) and/or the fourth (where present) antigen binding domain is HER2. In some aspects, the target antigen of the first and the second antigen binding domain is HER2. In some aspects, the target antigen of the first, the second, the third (where present) and the fourth (where present) antigen binding domain is HER2.

In some aspects, the first, the second, the third (where present) and/or the fourth (where present) antigen binding domain comprises a heavy chain variable region (VH) and a light chain variable region (VL).

In some aspects, the antigen binding domain is a humanized antigen binding domain (i.e. an antigen binding domain of a humanized antibody). In some aspects, the VH and/or the VL of the antigen binding domain is a humanized variable region. In some aspects, the VH and/or the VL of the antigen binding domain comprises an acceptor human framework, e.g. a human immunoglobulin framework or a human consensus framework.

The following aspects relate to the VH and VL of the first, the second, the third (where present) and/or the fourth (where present) antigen binding domain, wherein the antigen binding domain is capable of binding to HER2 (i.e. the target antigen of the antigen binding domain is HER2).

In some aspects, the VH of the antigen binding domain comprises a heavy chain complementarity determining region (HCDR) 1 of SEQ ID NO: 39, the HCDR 2 of SEQ ID NO: 40 and the HCDR 3 of SEQ ID NO: 41, and the VL of the antigen binding domain comprises a light chain complementarity determining region (LCDR) 1 of SEQ ID NO: 43, a LCDR 2 of SEQ ID NO: 44 and a LCDR 3 of SEQ ID NO: 45. In further aspects, the VH of the antigen binding domain comprises an amino acid sequence that is at least about 95%, 96%, 97%, 98%, 99% or 100% identical to the amino acid sequence of SEQ ID NO: 42, and/or the VL of the antigen binding domain comprises an amino acid sequence that is at least about 95%, 96%, 97%, 98%, 99% or 100% identical to the amino acid sequence of SEQ ID NO: 46.

In some aspects, the VH of the antigen binding domain comprises one or more heavy chain framework sequence (i.e. the FR1, FR2, FR3 and/or FR4 sequence) of the heavy chain variable region sequence of SEQ ID NO: 42. In some aspects, the VH of the antigen binding domain comprises an amino acid sequence that is at least about 95%, 96%, 97%, 98%, or 99% identical to the amino acid sequence of SEQ ID NO: 42. In some aspects, the VH of the antigen binding domain comprises an amino acid sequence that is at least about 95% identical to the amino acid sequence of SEQ ID NO: 42. In some aspects, the VH of the antigen binding domain comprises an amino acid sequence that is at least about 98% identical to the amino acid sequence of SEQ ID NO: 42. In certain aspects, a VH sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to HER2. In certain aspects, a total of 1 to 10 amino acids have been substituted, inserted and/or deleted in the amino acid sequence of SEQ ID NO: 42. In certain aspects, substitutions, insertions, or deletions occur in regions outside the CDRs (i.e., in the FRs). In some aspects, the VH of the antigen binding domain comprises the amino acid sequence of SEQ ID NO: 42. Optionally, the VH of the antigen binding domain comprises the amino acid sequence of SEQ ID NO: 42, including post-translational modifications of that sequence.

In some aspects, the VL of the antigen binding domain comprises one or more light chain framework sequence (i.e. the FR1, FR2, FR3 and/or FR4 sequence) of the light chain variable region sequence of SEQ ID NO: 46. In some aspects, the VL of the antigen binding domain comprises an amino acid sequence that is at least about 95%, 96%, 97%, 98%, or 99% identical to the amino acid sequence of SEQ ID NO: 46. In some aspects, the VL of the antigen binding domain comprises an amino acid sequence that is at least about 95% identical to the amino acid sequence of SEQ ID NO: 46. In some aspects, the VL of the antigen binding domain comprises an amino acid sequence that is at least about 98% identical to the amino acid sequence of SEQ ID NO: 46. In certain aspects, a VL sequence having at least 95%, 96%, 97%, 98%, or 99% identity contains substitutions (e.g., conservative substitutions), insertions, or deletions relative to the reference sequence, but an antigen binding domain comprising that sequence retains the ability to bind to HER2. In certain aspects, a total of 1 to 10 amino acids have been substituted, inserted and/or deleted in the amino acid sequence of SEQ ID NO: 46. In certain aspects, substitutions, insertions, or deletions occur in regions outside the CDRs (i.e., in the FRs). In some aspects, the VL of the antigen binding domain comprises the amino acid sequence of SEQ ID NO: 46. Optionally, the VL of the antigen binding domain comprises the amino acid sequence of SEQ ID NO: 42, including post-translational modifications of that sequence.

In some aspects, the VH of the antigen binding domain comprises an amino acid sequence that is at least about 95%, 96%, 97%, 98%, or 99% identical to the amino acid sequence of SEQ ID NO: 42, and the VL of the antigen binding domain comprises an amino acid sequence that is at least about 95%, 96%, 97%, 98%, or 99% identical to the amino acid sequence of SEQ ID NO: 46. In some aspects, the VH of the antigen binding domain comprises the amino acid sequence of SEQ ID NO: 42 and the VL of the antigen binding domain comprises the amino acid sequence of SEQ ID NO: 46.

In some aspects, the first, the second, the third (where present) and/or the fourth (where present) antigen binding domain is an antigen binding domain that binds to HER2 comprising a VH comprising the amino acid sequence of SEQ ID NO: 42 and a VL comprising the amino acid sequence of SEQ ID NO: 46. In some aspects, the first, the second, the third (where present) and/or the fourth (where present) antigen binding domain is an antigen binding domain that binds to HER2 comprising a VH sequence of SEQ ID NO: 42 and a VL sequence of SEQ ID NO: 46.

In some aspects, the first, the second, the third (where present) and/or the fourth (where present) antigen binding domain is an antigen binding domain that binds to HER2 comprising a VH comprising the heavy chain CDR sequences of the VH of SEQ ID NO: 42, and a VL comprising the light chain CDR sequences of the VL of SEQ ID NO: 46. In some aspects, said antigen binding domain comprises the HCDR1, HCDR2 and HCDR3 amino acid sequences of the VH of SEQ ID NO: 42 and the LCDR1, LCDR2 and LCDR3 amino acid sequences of the VL of SEQ ID NO: 46.

In some aspects, the VH of the antigen binding domain comprises the heavy chain CDR sequences of the VH of SEQ ID NO: 42 and a framework of at least 95%, 96%, 97%, 98% or 99% sequence identity to the framework sequence of the VH of SEQ ID NO: 42. In some aspects, the VH of the antigen binding domain comprises the heavy chain CDR sequences of the VH of SEQ ID NO: 42 and a framework of at least 95% sequence identity to the framework sequence of the VH of SEQ ID NO: 42. In some aspects, the VH of the antigen binding domain comprises the heavy chain CDR sequences of the VH of SEQ ID NO: 42 and a framework of at least 98% sequence identity to the framework sequence of the VH of SEQ ID NO: 42.

In some aspects, the VL of the antigen binding domain comprises the light chain CDR sequences of the VL of SEQ ID NO: 46 and a framework of at least 95%, 96%, 97%, 98% or 99% sequence identity to the framework sequence of the VL of SEQ ID NO: 46. In some aspects, the VL of the antigen binding domain comprises the light chain CDR sequences of the VL of SEQ ID NO: 46 and a framework of at least 95% sequence identity to the framework sequence of the VL of SEQ ID NO: 46. In some aspects, the VL of the antigen binding domain comprises the light chain CDR sequences of the VL of SEQ ID NO: 46 and a framework of at least 98% sequence identity to the framework sequence of the VL of SEQ ID NO: 46.

In some aspects, the first, the second, the third (where present) and/or the fourth (where present) antigen binding domain is an antigen binding domain, particularly a Fab molecule, that binds to HER2 comprising a VH sequence as in any of the aspects provided above, and a VL sequence as in any of the aspects provided above.

In some aspects, the first, the second, the third (where present) and the fourth (where present) antigen binding domain are each an antigen binding domain, particularly a Fab molecule, that binds to HER2 comprising a VH sequence as in any of the aspects provided above, and a VL sequence as in any of the aspects provided above.

### Fc Domain

According to the invention, the first and/or the second binding molecule may comprise an Fc domain.

The Fc domain of the binding molecule(s) consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

In some aspects, the first binding molecule comprises a first Fc domain composed of a first and a second subunit, and/or the second binding molecule comprises a second Fc domain composed of a first and a second subunit. In some aspects, the first binding molecule comprises a first Fc domain composed of a first and a second subunit, and the second binding molecule comprises a second Fc domain composed of a first and a second subunit. In some aspects, each of the binding molecules comprises not more than one Fc domain.

In some aspects, the Fc domain of the first and/or the second is an IgG Fc domain. In particular aspects, the Fc domain is an IgG₁ Fc domain. In other aspects, the Fc domain is an IgG₄ Fc domain. In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In further aspects, the Fc domain is a human Fc domain. In particular aspects, the Fc domain is a human IgG₁ Fc domain. An exemplary sequence of a human IgG₁ Fc region is given in SEQ ID NO: 59.

In some aspects, the Fc domain comprises a modification promoting the association of the first and the second subunit of the Fc domain. Fc domain modifications promoting heterodimerization are further described hereinbelow.

In some aspects, the Fc domain comprises one or more amino acid substitution that reduces binding to an Fc receptor and/or effector function. Fc domain modifications reducting Fc receptor binding and/or effector function are further described hereinbelow.

In some aspects, the first and the third (where present) antigen binding domain are each fused to a subunit of the first Fc domain, and/or the second and the fourth (where present) antigen binding domain are each fused to a subunit of the second Fc domain. In some aspects, the first and the third (where present) antigen binding domain are each fused at their C-terminus to the N-terminus of a subunit of the first Fc domain, and/or the second and the fourth (where present) antigen binding domain are each fused at their C-terminus to the N-terminus of a subunit of the second Fc domain.

### a) Fc Domain Modifications Promoting Heterodimerization

The binding molecule(s) according to the invention comprise a part of an effector domain, a complementing domain, and one or more antigen binding domains, which may be fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the binding molecule(s) in recombinant production, it will thus be advantageous to introduce in the Fc domain of the binding molecule(s) a modification promoting the association of the desired polypeptides.

Accordingly, in particular aspects, the Fc domain of the binding molecule(s) according to the invention comprises a modification promoting the association of the first and the second subunit of the Fc domain. The site of most extensive protein-protein interaction between the two subunits of a human IgG Fc domain is in the CH3 domain of the Fc domain. Thus, in some aspects, said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homdimers between the two first or the two second CH3 domains are formed).

In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in preferred aspects, in the CH3 domain of the first subunit of the Fc domain of the binding molecule(s) an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine(S), threonine (T), and valine (V).

The protuberance and cavity can be made by altering the nucleic acid encoding the polypeptides, e.g. by site-specific mutagenesis, or by peptide synthesis.

In specific aspects, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In some aspects, in the second subunit of the Fc domain additionally the threonine residue at position 366 is replaced with a serine residue (T366S) and the leucine residue at position 368 is replaced with an alanine residue (L368A) (numberings according to Kabat EU index).

In yet further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In particular aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index). Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In some aspects, the heterodimerization approach described in EP 1870459, is used alternatively. This approach is based on the introduction of charged amino acids with opposite charges at specific amino acid positions in the CH3/CH3 domain interface between the two subunits of the Fc domain. A particular aspect for the binding molecule(s) of the invention are amino acid mutations R409D; K370E in one of the two CH3 domains (of the Fc domain) and amino acid mutations D399K; E357K in the other one of the CH3 domains of the Fc domain (numbering according to Kabat EU index).

In some aspects, the binding molecule(s) of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In some aspects, the binding molecule(s) of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said binding molecule(s) comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In some aspects, the heterodimerization approach described in WO 2013/157953 is used alternatively. In some aspects, a first CH3 domain comprises amino acid mutation T366K and a second CH3 domain comprises amino acid mutation L351D (numberings according to Kabat EU index). In further aspects, the first CH3 domain comprises further amino acid mutation L351K. In further aspects, the second CH3 domain comprises further an amino acid mutation selected from Y349E, Y349D and L368E (particularly L368E) (numberings according to Kabat EU index).

In some aspects, the heterodimerization approach described in WO 2012/058768 is used alternatively. In some aspects, a first CH3 domain comprises amino acid mutations L351Y, Y407A and a second CH3 domain comprises amino acid mutations T366A, K409F. In further aspects, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F4051, F405M, F405T, F405S, F405V or F405W, c) N390R, N390K or N390D, f) K392V, K392M, K392R, K392L, K392F or K392E (numberings according to Kabat EU index). In further aspects, a first CH3 domain comprises amino acid mutations L351Y, Y407A and a second CH3 domain comprises amino acid mutations T366V, K409F. In further aspects, a first CH3 domain comprises amino acid mutation Y407A and a second CH3 domain comprises amino acid mutations T366A, K409F. In further aspects, the second CH3 domain further comprises amino acid mutations K392E, T411E, D399R and S400R (numberings according to Kabat EU index).

In some aspects, the heterodimerization approach described in WO 2011/143545 is used alternatively, e.g. with the amino acid modification at a position selected from the group consisting of 368 and 409 (numbering according to Kabat EU index).

In some aspects, the heterodimerization approach described in WO 2011/090762, which also uses the knobs-into-holes technology described above, is used alternatively. In some aspects, a first CH3 domain comprises amino acid mutation T366W and a second CH3 domain comprises amino acid mutation Y407A. In some aspects, a first CH3 domain comprises amino acid mutation T366Y and a second CH3 domain comprises amino acid mutation Y407T (numberings according to Kabat EU index).

In some aspects, the binding molecule(s) or its Fc domain is of IgG₂ subclass and the heterodimerization approach described in WO 2010/129304 is used alternatively.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In some such aspects, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In further aspects, the first CH3 domain further comprises amino acid substitution of K409 or R409 with a negatively charged amino acid (e.g. glutamic acid (E), or aspartic acid (D), particularly K409D or R409D). In further aspects, the first CH3 domain further or alternatively comprises amino acid substitution of K439 and/or K370 with a negatively charged amino acid (e.g. glutamic acid (E), or aspartic acid (D)) (all numberings according to Kabat EU index).

In some aspects, the heterodimerization approach described in WO 2007/147901 is used alternatively. In some aspects, a first CH3 domain comprises amino acid mutations K253E, D282K, and K322D and a second CH3 domain comprises amino acid mutations D239K, E240K, and K292D (numberings according to Kabat EU index).

In some aspects, the heterodimerization approach described in WO 2007/110205 can be used alternatively.

In some aspects, the first subunit of the Fc domain comprises amino acid substitutions K392D and K409D, and the second subunit of the Fc domain comprises amino acid substitutions D356K and D399K (numbering according to Kabat EU index).

b) Fc Domain Modifications Reducing Fc Receptor Binding and/or Effector Function

The Fc domain confers to the binding molecule(s) favorable pharmacokinetic properties, including a long serum half-life which contributes to good accumulation in the target tissue and a favorable tissue-blood distribution ratio. At the same time it may, however, lead to undesirable targeting of the binding molecule(s) to cells expressing Fc receptors rather than to the preferred antigen-bearing cells. Moreover, the co-activation of Fc receptor signaling pathways may lead to cytokine release which may result in excessive activation of cytokine receptors and severe side effects upon systemic administration. Activation of (Fc receptor-bearing) immune cells other than T cells may even reduce efficacy of the pair of binding molecules due to the potential destruction of T cells e.g. by NK cells.

Accordingly, in particular aspects, the Fc domain of the binding molecule(s) according to the invention exhibits reduced binding affinity to an Fc receptor and/or reduced effector function, as compared to a native IgG₁ Fc domain. In some such aspects, the Fc domain (or the binding molecule comprising said Fc domain) exhibits less than 50%, particularly less than 20%, more particularly less than 10% and most particularly less than 5% of the binding affinity to an Fc receptor, as compared to a native IgG₁ Fc domain (or a binding molecule comprising a native IgG₁ Fc domain), and/or less than 50%, particularly less than 20%, more particularly less than 10% and most particularly less than 5% of the effector function, as compared to a native IgG₁ Fc domain domain (or a binding molecule comprising a native IgG₁ Fc domain). In some aspects, the Fc domain domain (or the binding molecule comprising said Fc domain) does not substantially bind to an Fc receptor and/or induce effector function. In particular aspects, the Fc receptor is an Fcγ receptor. In some aspects, the Fc receptor is a human Fc receptor. In some aspects, the Fc receptor is an activating Fc receptor. In specific aspects, the Fc receptor is an activating human Fcγ receptor, more specifically human FcγRIIIa, FcγRI or FcγRIIa, most specifically human FcγRIIIa. In some aspects, the effector function is one or more selected from the group of CDC, ADCC, ADCP, and cytokine secretion. In particular aspects, the effector function is ADCC. In some aspects, the Fc domain domain exhibits substantially similar binding affinity to neonatal Fc receptor (FcRn), as compared to a native IgG₁ Fc domain domain. Substantially similar binding to FcRn is achieved when the Fc domain (or the binding molecule comprising said Fc domain) exhibits greater than about 70%, particularly greater than about 80%, more particularly greater than about 90% of the binding affinity of a native IgG₁ Fc domain (or the binding molecule comprising a native IgG₁ Fc domain) to FcRn.

In certain aspects, the Fc domain is engineered to have reduced binding affinity to an Fc receptor and/or reduced effector function, as compared to a non-engineered Fc domain. In particular aspects, the Fc domain of the binding molecule(s) comprises one or more amino acid mutation that reduces the binding affinity of the Fc domain to an Fc receptor and/or effector function. Typically, the same one or more amino acid mutation is present in each of the two subunits of the Fc domain. In some aspects, the amino acid mutation reduces the binding affinity of the Fc domain to an Fc receptor. In some aspects, the amino acid mutation reduces the binding affinity of the Fc domain to an Fc receptor by at least 2-fold, at least 5-fold, or at least 10-fold. In aspects where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In some aspects, the binding molecule(s) comprising an engineered Fc domain exhibits less than 20%, particularly less than 10%, more particularly less than 5% of the binding affinity to an Fc receptor as compared to a binding molecule comprising a non-engineered Fc domain. In particular aspects, the Fc receptor is an Fcγ receptor. In some aspects, the Fc receptor is a human Fc receptor. In some aspects, the Fc receptor is an activating Fc receptor. In specific aspects, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In some aspects, binding affinity to neonatal Fc receptor (FcRn) is not reduced. Substantially similar binding to FcRn, i.e. preservation of the binding affinity of the Fc domain to said receptor, is achieved when the Fc domain (or the binding molecule comprising said Fc domain) exhibits greater than about 70% of the binding affinity of a non-engineered form of the Fc domain (or the binding molecule comprising said non-engineered form of the Fc domain) to FcRn. The Fc domain, or binding molecule(s) of the invention comprising said Fc domain, may exhibit greater than about 80% and even greater than about 90% of such affinity. In certain aspects, the Fc domain of the binding molecule(s) is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In some aspects, the reduced effector function is one or more selected from the group of reduced CDC, reduced ADCC, reduced ADCP, and reduced cytokine secretion. In particular aspects, the reduced effector function is reduced ADCC. In some aspects, the reduced ADCC is less than 20% of the ADCC induced by a non-engineered Fc domain (or a binding molecule comprising a non-engineered Fc domain).

In some aspects, the amino acid mutation that reduces the binding affinity of the Fc domain to an Fc receptor and/or effector function is an amino acid substitution. In some aspects, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In some such aspects, the Fc domain is an IgG₁ Fc domain, particularly a human IgG₁ Fc domain. In some aspects, the Fc domain comprises an amino acid substitution at position P329. In a more specific aspect, the amino acid substitution is P329A or P329G, particularly P329G (numberings according to Kabat EU index). In some aspects, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in particular aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In some such aspects, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some aspects, the Fc domain of the binding molecule(s) of the invention is an IgG₄ Fc domain, particularly a human IgG₄ Fc domain. In some aspects, the IgG₄ Fc domain comprises an amino acid substitution at position S228, specifically the amino acid substitution S228P (numberings according to Kabat EU index). To further reduce its binding affinity to an Fc receptor and/or its effector function, in some aspects, the IgG₄ Fc domain comprises an amino acid substitution at position L235, specifically the amino acid substitution L235E (numberings according to Kabat EU index). In some aspects, the IgG₄ Fc domain comprises an amino acid substitution at position P329, specifically the amino acid substitution P329G (numberings according to Kabat EU index). In a preferred aspect, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In particular aspects, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain aspects, N-glycosylation of the Fc domain has been eliminated. In some such aspects, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. Alternatively, binding affinity of Fc domains or binding molecule(s) comprising an Fc domain for Fc receptors may be evaluated using cell lines known to express particular Fc receptors, such as human NK cells expressing FcγIIIa receptor.

Effector function of an Fc domain, or a binding molecule comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some aspects, binding of the Fc domain to a complement component, specifically to C1q, is reduced. Accordingly, in some aspects wherein the Fc domain is engineered to have reduced effector function, said reduced effector function includes reduced CDC. C1q binding assays may be carried out to determine whether the Fc domain, or the binding molecule comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18 (12): 1759-1769 (2006); WO 2013/120929).

### Configuration of the Binding Molecules

The binding molecule(s) according to the present invention may have various molecular configurations, i.e. the domains of the binding molecule(s) may be linked to each other in different ways.

In particular aspects, the binding molecule(s) comprise an Fc domain composed of a first and a second subunit, and (i) the antigen binding domain(s) is fused at its C-terminus to the N-terminus of one of the subunits of the Fc domain, (ii) the part of the effector domain is fused at its N-terminus to the C-terminus of one of the subunits of the Fc domain, and (iii) the complementing domain is fused at its N-terminus to the C-terminus of the part of the effector domain. In some such aspects, the effector domain is an antigen binding domain, particularly an anti-CD3 antigen binding domain.

In some aspects, the antigen binding domain(s) is a Fab molecule and is fused at the C-terminus of its heavy chain to the N-terminus of one of the subunits of the Fc domain. In some aspects, the part of the effector domain comprises (or consists of) a VH or VL and is fused at its N-terminus to the C-terminus of one of the subunits of the Fc domain. In some aspects, the complementing domain comprises (or consists of) a VH or VL and is fused at its N-terminus to the C-terminus of the part of the effector domain. In some such aspects, the effector domain is an antigen binding domain, particularly an anti-CD3 antigen binding domain.

In some aspects,
the first binding molecule comprises
(i) a first and optionally a third antigen binding domain, (ii) a first Fc domain composed of a first and a second subunit, (iii) a first part of an effector domain, and (iv) a first complementing domain,
wherein
(a) the first and the third (where present) antigen binding domain is fused at its C-terminus to the N-terminus of one of the subunits of the first Fc domain,
(b) the first part of the effector domain is fused at its N-terminus to the C-terminus of one of the subunits of the first Fc domain,
(c) the first complementing domain is fused at its N-terminus to the C-terminus of the first part of the effector domain; and
the second binding molecule comprises
(i) a second and optionally a fourth antigen binding domain, (ii) a second Fc domain composed of a first and a second subunit, (iii) a second part of an effector domain, and (iv) a second complementing domain,
wherein
(a) the second and the fourth (where present) antigen binding domain is fused at its C-terminus to the N-terminus of one of the subunits of the second Fc domain,
(b) the second part of the effector domain is fused at its N-terminus to the C-terminus of one of the subunits of the second Fc domain,
(c) the second complementing domain is fused at its N-terminus to the C-terminus of the second part of the effector domain.

In some such aspects, the effector domain is an antigen binding domain, particularly an anti-CD3 antigen binding domain.

In some aspects,
the first binding molecule comprises
(i) a first and optionally a third antigen binding domain, (ii) a first Fc domain composed of a first and a second subunit, (iii) a first part of an effector domain, and (iv) a first complementing domain,
wherein
(a) the first and the third (where present) antigen binding domain is fused at its C-terminus to the N-terminus of one of the subunits of the first Fc domain,
(b) the first part of the effector domain comprises (or consists of) a VH and is fused at its N-terminus to the C-terminus of one of the subunits of the first Fc domain,
(c) the first complementing domain comprises (or consists of) a VL and is fused at its N-terminus to the C-terminus of the first part of the effector domain; and the second binding molecule comprises
(i) a second and optionally a fourth antigen binding domain, (ii) a second Fc domain composed of a first and a second subunit, (iii) a second part of an effector domain, and (iv) a second complementing domain,
wherein
(a) the second and the fourth (where present) antigen binding domain is fused at its C-terminus to the N-terminus of one of the subunits of the second Fc domain,
(b) the second part of the effector domain comprises (or consists of) a VL and is fused at its N-terminus to the C-terminus of one of the subunits of the second Fc domain,
(c) the second complementing domain comprises (or consists of) a VH and is fused at its N-terminus to the C-terminus of the second part of the effector domain.

In some such aspects, the effector domain is an anti-CD3 antigen binding domain, particularly an anti-CD3 Fv molecule.

In some aspects,
the first binding molecule comprises
(i) a first and optionally a third antigen binding domain, (ii) a first Fc domain composed of a first and a second subunit, (iii) a first part of an effector domain, and (iv) a first complementing domain,
wherein
(a) the first antigen binding domain is a Fab molecule and is fused at its C-terminus to the N-terminus of the first subunit of the first Fc domain,
(b) the third antigen binding domain (where present) is a Fab molecule and is fused at its C-terminus to the N-terminus of the first subunit of the first Fc domain,
(c) the first part of the effector domain comprises (or consists of) a VH and is fused at its N-terminus to the C-terminus of the first or the second subunit of the first Fc domain,
(d) the first complementing domain comprises (or consists of) a VL and is fused at its N-terminus to the C-terminus of the first part of the effector domain; and the second binding molecule comprises
(i) a second and optionally a fourth antigen binding domain, (ii) a second Fc domain composed of a first and a second subunit, (iii) a second part of an effector domain, and (iv) a second complementing domain,
wherein
(a) the second antigen binding domain is a Fab molecule and is fused at its C-terminus to the N-terminus of the first subunit of the second Fc domain,
(b) the fourth antigen binding domain (where present) is a Fab molecule and is fused at its C-terminus to the N-terminus of the first subunit of the second Fc domain,
(c) the second part of the effector domain comprises (or consists of) a VL and is fused at its N-terminus to the C-terminus of the first or the second subunit of the second Fc domain,
(d) the second complementing domain comprises (or consists of) a VH and is fused at its N-terminus to the C-terminus of the second part of the effector domain.

In some such aspects, the effector domain is an anti-CD3 antigen binding domain, particularly an anti-CD3 Fv molecule.

In some aspects, the first binding molecule consists of the first antigen binding domain, the first Fc domain, the first part of the effector domain, the first complementing domain, and optionally one or more peptide linkers, and/or the second binding molecule consists of the second antigen binding domain, the second Fc domain, the second part of the effector domain, the second complementing domain, and optionally one or more peptide linkers. In some aspects, the first binding molecule consists of the first antigen binding domain, the third antigen binding domain, the first Fc domain, the first part of the effector domain, the first complementing domain, and optionally one or more peptide linkers, and/or the second binding molecule consists of the second antigen binding domain, the fourth antigen binding domain, the second Fc domain, the second part of the effector domain, the second complementing domain, and optionally one or more peptide linkers.

### Peptide Linkers

The domains of the binding molecule(s) according to the invention (antigen binding domain, effector domain, complementing domain, Fc domain . . . ) may be fused to each other directly or through one or more peptide linker.

Peptides linkers comprise one or more amino acids, typically about 2-20 amino acids. Peptide linkers are known in the art and are described herein. Suitable, non-immunogenic peptide linkers include, for example, (G₄S)ₙ, (SG₄)ₙ, G₄(SG₄)ₙ or (G₄S)ₙG₅ peptide linkers. “n” is generally an integer from 1 to 10, typically from 2 to 4. In some aspects, said peptide linker has a length of at least 5 amino acids, in some aspects a length of 5 to 100, in further aspects of 10 to 50 amino acids. In some aspects, said peptide linker is (GxS)ₙ or (GxS)ₙGₘ with G=glycine, S=serine, and (x=3, n=3, 4, 5 or 6, and m=0, 1, 2 or 3) or (x=4, n=1, 2, 3, 4 or 5 and m=0, 1, 2, 3, 4 or 5), in some aspects x=4 and n=2 or 3, in a further aspect x=4 and n=2, in yet a further aspect x=4, n=1 and m=5. In some aspects, said peptide linker is (G₄S)₂. (SEQ ID NO: 55). In other aspects, said peptide linker is (G₄S)G₅. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In some aspects, the first and the third (where present) antigen binding domain are each fused to one of the subunits of the first Fc domain via an immunoglobulin hinge region, and/or the second and the fourth (where present) antigen binding domain are each fused to one of the subunits of the second Fc domain via an immunoglobulin hinge region.

In some aspects, the first part of the effector domain and the first Fc domain, and/or the second part of the effector domain and the second Fc domain are fused via a peptide linker. In some aspects, the first part of the effector domain is fused at its N-terminus to the C-terminus of a subunit of the first Fc domain via a peptide linker, and/or the second part of the effector domain is fused at its N-terminus to the C-terminus of a subunit of the second Fc domain via a peptide linker. An exemplary peptide linker suitable for fusing the part of the effector domain to (the C-terminus of) a subunit of the Fc domain is (G₄S)₂. (SEQ ID NO: 55). Accordingly, in some aspects, the first part of the effector domain is fused at its N-terminus to the C-terminus of a subunit of the first Fc domain via a peptide linker comprising the sequence (G₄S)₂. (SEQ ID NO: 55), and/or the second part of the effector domain is fused at its N-terminus to the C-terminus of a subunit of the second Fc domain via a peptide linker comprising the sequence (G₄S)₂. (SEQ ID NO: 55). In particular aspects, said peptide linker consists of the sequence (G₄S)₂. (SEQ ID NO: 55).

In some aspects, the first part of the effector domain and the first complementing domain, and/or the second part of the effector domain and the second complementing domain are fused via a peptide linker. In some aspects, the first part of the effector domain is fused at its C-terminus to the N-terminus of the first complementing domain via a peptide linker, and/or the second part of the effector domain is fused at its C-terminus to the N-terminus of the second complementing domain via a peptide linker. An exemplary peptide linker suitable for fusing the complementing domain to (the C-terminus of) the part of the effector domain is (G₄S)₃GGSGG (SEQ ID NO: 56). Accordingly, in some aspects, the first part of the effector domain is fused at its C-terminus to the N-terminus of the first complementing domain via a peptide linker comprising the sequence of SEQ ID NO: 56, and/or the second part of the effector domain is fused at its C-terminus to the N-terminus of the second complementing domain via a peptide linker comprising the sequence of SEQ ID NO: 56. In particular aspects, said peptide linker consists of the sequence of SEQ ID NO: 56.

### Binding Molecule

The inventions also provides a binding molecule that forms part of the pair of binding molecules of the invention. Said binding molecule may incorporate, singly or in combination, any of the features described above and herein for the pair of binding molecules (unless the context dictates otherwise).

### Polynucleotides

The invention further provides an isolated polynucleotide encoding the pair of binding molecule of the invention. The invention also provides an isolated polynucleotide encoding a binding molecule that forms part of the pair of binding molecules of the inventions. Said isolated polynucleotide may be a single polynucleotide or a plurality of polynucleotides.

The polynucleotides encoding binding molecule(s) of the invention may be expressed as a single polynucleotide that encodes the entire pair of binding molecules or binding molecule or as multiple (e.g., two or more) polynucleotides that are co-expressed. Polypeptides encoded by polynucleotides that are co-expressed may associate through, e.g., disulfide bonds or other means to form a functional binding molecule. For example, the light chain portion of an antigen binding domain may be encoded by a separate polynucleotide from the portion of the binding molecule comprising the heavy chain of the antigen binding domain. When co-expressed, the heavy chain polypeptides will associate with the light chain polypeptides to form the antigen binding domain.

In another example, the portion of the binding molecule comprising one of the two Fc domain subunits and optionally (part of) one or more Fab molecules could be encoded by a separate polynucleotide from the portion of the binding molecule comprising the other of the two Fc domain subunits and optionally (part of) a Fab molecule. When co-expressed, the Fc domain subunits will associate to form the Fc domain.

In some aspects, the isolated polynucleotide encodes the entire pair of binding molecules or entire binding molecule according to the invention as described herein. In other aspects, the isolated polynucleotide encodes a polypeptide comprised in the pair of binding molecules or the binding molecule according to the invention as described herein.

In certain aspects, the polynucleotide or nucleic acid is DNA. In other aspects, a polynucleotide of the present invention is RNA, for example, in the form of messenger RNA (mRNA). RNA of the present invention may be single stranded or double stranded.

### Recombinant Methods

Binding molecules of the invention may be obtained, for example, by solid-state peptide synthesis (e.g. Merrifield solid phase synthesis) or recombinant production. For recombinant production one or more polynucleotide encoding the binding molecule(s), e.g., as described above, is isolated and inserted into one or more vectors for further cloning and/or expression in a host cell. Such polynucleotide may be readily isolated and sequenced using conventional procedures. In one aspect, a vector, particularly an expression vector, comprising the polynucleotide (i.e. a single polynucleotide or a plurality of polynucleotides) of the invention is provided. Methods which are well known to those skilled in the art can be used to construct expression vectors containing the coding sequence of an binding molecule along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N. Y (1989). The expression vector can be part of a plasmid, virus, or may be a nucleic acid fragment. The expression vector includes an expression cassette into which the polynucleotide encoding the binding molecule(s) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the binding molecule(s) of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible by tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions may be associated with additional coding regions which encode secretory or signal peptides, which direct the secretion of a polypeptide encoded by a polynucleotide of the present invention. For example, if secretion of the binding molecule is desired, DNA encoding a signal sequence may be placed upstream of the nucleic acid encoding the binding molecule or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain aspects, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the binding molecule may be included within or at the ends of the binding molecule (fragment) encoding polynucleotide.

In some aspects, a host cell comprising a polynucleotide (i.e. a single polynucleotide or a plurality of polynucleotides) of the invention is provided. In certain aspects, a host cell comprising a vector of the invention is provided. The polynucleotides and vectors may incorporate any of the features, singly or in combination, described herein in relation to polynucleotides and vectors, respectively. In some such aspects, a host cell comprises (e.g. has been transformed or transfected with) one or more vector comprising one or more polynucleotide that encodes (part of) a pair of binding molecules or binding molecule of the invention. As used herein, the term “host cell” refers to any kind of cellular system which can be engineered to generate the binding molecule(s) of the invention or fragments thereof. Host cells suitable for replicating and for supporting expression of binding molecules (e.g. antibodies) are well known in the art. Such cells may be transfected or transduced as appropriate with the particular expression vector and large quantities of vector containing cells can be grown for seeding large scale fermenters to obtain sufficient quantities of the binding molecule(s) for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In some aspects, the host cell is a eukaryotic cell, particularly a mammalian cell, such as a Chinese Hamster Ovary (CHO) cell, a human embryonic kidney (HEK) cell or a lymphoid cell (e.g., Y0, NS0, Sp20 cell). In some aspects, the host cell is not a cell within a human body.

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one aspect, a method of producing a pair of binding molecules according to the invention is provided, wherein the method comprises culturing a host cell comprising a polynucleotide encoding the pair of binding molecules, as provided herein, under conditions suitable for expression of the pair of binding molecules, and optionally recovering the pair of binding molecules from the host cell (or host cell culture medium). Similarly, in one aspect, a method of producing a binding molecule according to the invention is provided, wherein the method comprises culturing a host cell comprising a polynucleotide encoding the binding molecule, as provided herein, under conditions suitable for expression of the binding molecule, and optionally recovering the binding molecule from the host cell (or host cell culture medium).

Binding molecule(s) prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification, an antibody, ligand, receptor or antigen can be used to which the binding molecule binds. For example, for affinity chromatography purification of binding molecules of the invention, a matrix with protein A or protein G may be used. Sequential Protein A or G affinity chromatography and size exclusion chromatography can be used to isolate a binding molecule essentially as described in the Examples. The purity of the binding molecule can be determined by any of a variety of well-known analytical methods including gel electrophoresis, high pressure liquid chromatography, and the like.

### Compositions, Formulations, and Routes of Administration

In a further aspect, the invention provides pharmaceutical compositions comprising the pair of binding molecules or the binding molecule provided herein, e.g., for use in any of the below therapeutic methods. In some aspects, a pharmaceutical composition comprises a pair of binding molecule or a binding molecule according to the invention and a pharmaceutically acceptable carrier. In some aspects, a pharmaceutical composition comprises a pair of binding molecules or a binding molecule according to the invention and at least one additional therapeutic agent, e.g., as described below. The binding molecules that form part of the pair of binding molecules may be comprised in one and the same pharmaceutical composition, or in separate pharmaceutical compositions (i.e. each binding molecule that forms part of the pair of binding molecules is in a separate pharmaceutical composition). Typically, they would be provided in separate pharmaceutical formulations, to minimize the risk of the first and the second part of the effector domain associating with each other within the pharmaceutical composition. Thus, in particular aspects, the invention provides a first pharmaceutical composition comprising the first binding molecule and a pharmaceutically acceptable carrier, and a second pharmaceutical composition comprising the second binding molecule and a pharmaceutically acceptable carrier. In other aspects, the invention provides a pharmaceutical composition comprising the first and the second binding molecule and a pharmaceutically acceptable carrier.

Further provided is a method of producing a pair of binding molecules or binding molecule of the invention in a form suitable for administration in vivo, the method comprising (a) obtaining an pair of binding molecules or binding molecule according to the invention, and (b) formulating the pair of binding molecules or binding molecule with at least one pharmaceutically acceptable carrier, whereby a preparation of pair of binding molecules or binding molecule is formulated for administration in vivo.

Pharmaceutical compositions of the present invention comprise an effective amount of binding molecule(s) dissolved or dispersed in a pharmaceutically acceptable carrier. The phrase “pharmaceutically acceptable” refers to molecular entities and compositions that are generally nontoxic to recipients at the dosages and concentrations employed, i.e. do not produce an adverse, allergic or other untoward reaction when administered to an animal, such as, for example, a human, as appropriate. The preparation of a pharmaceutical composition that contains a binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

A pair of binding molecules or binding molecule of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. In some aspects, the binding molecule(s) of the invention are (to be) administered intravenously.

Parenteral compositions include those designed for administration by injection, e.g. subcutaneous, intradermal, intralesional, intravenous, intraarterial intramuscular, intrathecal or intraperitoneal injection. For injection, the binding molecule(s) of the invention may be formulated in aqueous solutions, particularly in physiologically compatible buffers such as Hanks' solution, Ringer's solution, or physiological saline buffer. The solution may contain formulatory agents such as suspending, stabilizing and/or dispersing agents. Alternatively, the binding molecule(s) may be in powder form for constitution with a suitable vehicle, e.g., sterile pyrogen-free water, before use. Sterile injectable solutions are prepared by incorporating the binding molecule(s) of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular aspects, prolonged absorption of an injectable composition can be brought about by the use in the compositions of agents delaying absorption, such as, for example, aluminum monostearate, gelatin or combinations thereof.

In addition to the compositions described previously, the binding molecule(s) may also be formulated as a depot preparation. Such long acting formulations may be administered by implantation (for example subcutaneously or intramuscularly) or by intramuscular injection. Thus, for example, the binding molecule(s) may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the binding molecule(s) of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The binding molecule(s) may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaccous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the binding molecule(s) provided herein may be used in therapeutic methods. Binding molecule(s) of the invention may be used as immunotherapeutic agents, for example in the treatment of cancers.

For use in therapeutic methods, binding molecule(s) of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In therapeutic methods, the first and the second binding molecule are used in combination. As used herein, “combination” (and grammatical variations thereof such as “combine” or “combining”) encompasses combinations of the first and the second binding molecules wherein the first and the second binding molecule are in the same or in different containers, in the same or in different pharmaceutical formulations, administered together or separately, administered simultaneously or sequentially, in any order, and administered by the same or by different routes, provided that the first and the second binding molecule, via their antigen binding domain, can simultaneously bind to their target antigens on the surface of a cell. For example “combining” the first and the second binding molecule may mean first administering the first binding molecule in a particular pharmaceutical formulation, followed by administration of the second binding molecule in another pharmaceutical formulation, or vice versa.

The first and the second binding molecule may be administered in any suitable manner known in the art. In some aspects, the first and the second binding molecule are administered sequentially (at different times). In other aspects, the first and the second binding molecule are administered concurrently (at the same time). In some aspects, first and the second binding molecule are in separate compositions. In some aspects, the first and the second binding molecule are in the same composition.

In one aspect, the pair of binding molecules or binding molecule of the invention for use as a medicament is provided. In further aspects, the pair of binding molecules or binding molecule of the invention for use in treating a disease are provided. In certain aspects, the pair of binding molecules or binding molecule of the invention for use in a method of treatment are provided. In one aspect, the invention provides a pair or binding molecules or binding molecule of the invention for use in the treatment of a disease in an individual in need thereof. In one aspect, the invention provides a pair of binding molecules for use in a method of treating an individual having a disease comprising administering to the individual an effective amount of the pair of binding molecules. In certain aspects, the disease is a proliferative disorder. In certain aspects, the disease is cancer. In some aspects, the cancer is a solid tumor cancer. In some aspects, the cancer is a cancer expressing the target antigen(s) of the pair of binding molecules or binding molecule. In some aspects (specifically if the antigen binding domain(s) of the first and/or the second binding molecule are capable of binding HER2), the cancer is a HER2-expressing cancer. In specific aspects, the cancer is breast cancer. In certain aspects, the method further comprises administering to the individual an effective amount of at least one additional therapeutic agent, e.g., an anti-cancer agent if the disease to be treated is cancer. In a further aspect, the invention provides a pair of binding molecules of the invention for use in inducing lysis of a target cell, particularly a cancer cell. In one aspect, the invention provides a pair of binding molecules of the invention for use in a method of inducing lysis of a target cell, particularly a cancer cell, in an individual comprising administering to the individual an effective amount of the pair of binding molecules to induce lysis of a target cell. An “individual” according to any of the above aspects is a mammal, preferably a human.

In a further aspect, the invention provides for the use of a pair of binding molecules or binding molecule of the invention in the manufacture or preparation of a medicament. In one aspect, the medicament is for the treatment of a disease in an individual in need thereof. In further aspects, the medicament is for use in a method of treating a disease comprising administering to an individual having the disease an effective amount of the medicament. In certain aspects, the disease is a proliferative disorder. In certain aspects, the disease is cancer. In some aspects, the cancer is a solid tumor cancer. In some aspects, the cancer is a cancer expressing the target antigen(s) of the pair of binding molecules or binding molecule. In some aspects (specifically if the antigen binding domain(s) of the first and/or the second binding molecule are capable of binding HER2), the cancer is a HER2-expressing cancer. In specific aspects, the cancer is breast cancer. In some aspects, the method further comprises administering to the individual an effective amount of at least one additional therapeutic agent, e.g., an anti-cancer agent if the disease to be treated is cancer. In further aspects, the medicament is for inducing lysis of a target cell, particularly a cancer cell. In still further aspects, the medicament is for use in a method of inducing lysis of a target cell, particularly a cancer cell, in an individual comprising administering to the individual an effective amount of the medicament to induce lysis of a target cell. An “individual” according to any of the above aspects may be a mammal, preferably a human.

In a further aspect, the invention provides a method for treating a disease. In one aspect, the method comprises administering to an individual having such disease an effective amount of a pair of binding molecules of the invention. In some aspects, one or more composition is administered to said individual, comprising the pair of binding molecules of the invention in a pharmaceutically acceptable form. In certain aspects, the disease is a proliferative disorder. In certain aspects, the disease is cancer. In some aspects, the cancer is a solid tumor cancer. In some aspects, the cancer is a cancer expressing the target antigen(s) of the pair of binding molecules or binding molecule. In some aspects (specifically if the antigen binding domain(s) of the first and/or the second binding molecule are capable of binding HER2), the cancer is a HER2-expressing cancer. In specific aspects, the cancer is breast cancer. In certain aspects, the method further comprises administering to the individual an effective amount of at least one additional therapeutic agent, e.g., an anti-cancer agent if the disease to be treated is cancer. An “individual” according to any of the above aspects may be a mammal, preferably a human.

In a further aspect, the invention provides a method for inducing lysis of a target cell. In some aspects, the target cell is a cell expressing the target antigen(s) of the pair of binding molecules or binding molecule. In some aspects (specifically if the antigen binding domain(s) of the first and/or the second binding molecule are capable of binding HER2), the target cell is a HER2-expressing cell. In some aspects, the method comprises contacting a target cell with a pair of binding molecules of the invention in the presence of a T cell, particularly a cytotoxic T cell. In a further aspect, a method for inducing lysis of a target cell in an individual is provided. In some aspects, the target cell is a cell expressing the target antigen(s) of the pair of binding molecules or binding molecule. In some aspects (specifically if the antigen binding domain(s) of the first and/or the second binding molecule are capable of binding HER2), the target cell is a HER2-expressing cell. In some such aspects, the method comprises administering to the individual an effective amount of the pair of binding molecules of the invention to induce lysis of a target cell. In one aspect, an “individual” is a human.

A skilled artisan readily recognizes that in many cases the pair of binding molecules may not provide a cure but may only provide partial benefit. In some aspects, a physiological change having some benefit is also considered therapeutically beneficial. Thus, in some aspects, an amount of the pair of binding molecules that provides a physiological change is considered an “effective amount”. The subject, patient, or individual in need of treatment is typically a mammal, more specifically a human.

In some aspects, an effective amount of a pair of binding molecules of the invention is administered to an individual for the treatment of disease.

For the prevention or treatment of disease, the appropriate dosage of a pair of binding molecules of the invention (when used alone or in combination with one or more other additional therapeutic agents) will depend on the type of disease to be treated, the route of administration, the body weight of the patient, the type of binding molecules, the severity and course of the disease, whether the pair of binding molecules is administered for preventive or therapeutic purposes, previous or concurrent therapeutic interventions, the patient's clinical history and response to the pair of binding molecules, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The pair of binding molecules is suitably administered to the patient at one time or over a series of treatments. Depending on the type and severity of the disease, about 1 μg/kg to 15 mg/kg (e.g. 0.1 mg/kg-10 mg/kg) per binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g. every week or every three weeks (e.g. such that the patient receives from about two to about twenty, or e.g. about six doses of the pair of binding molecules). An initial higher loading dose, followed by one or more lower doses may be administered. However, other dosage regimens may be useful. The progress of this therapy is easily monitored by conventional techniques and assays.

The pair of binding molecules of the invention will generally be used in an amount effective to achieve the intended purpose. For use to treat or prevent a disease condition, the pair of binding molecules of the invention, or pharmaceutical compositions thereof, are administered or applied in an effective amount.

For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art.

Dosage amount and interval may be adjusted individually to provide plasma levels of the binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

An effective dose of the pair of binding molecules of the invention will generally provide therapeutic benefit without causing substantial toxicity. Toxicity and therapeutic efficacy of a pair of binding molecules can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Pairs of binding molecules that exhibit large therapeutic indices are preferred. In some aspects, the pair of binding molecules according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with a pair of binding molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The binding molecules that form the pair of binding molecules (i.e. the first and the second binding molecule) may be administered in a combined administration (where the binding molecules are included in the same or separate compositions), or in separate administrations, in which case, administration of the first binding molecule can occur prior to, simultaneously, and/or following, administration of the second binding molecule.

The pair of binding molecules of the invention may be administered in combination with one or more other agents in therapy. For instance, a pair of binding molecules of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular disease being treated, preferably those with complementary activities that do not adversely affect each other. In certain aspects, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In certain aspects, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of the pair of binding molecules used, the type of disorder or treatment, and other factors discussed above. The pair of binding molecules is generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the pair of binding molecules of the invention can occur prior to, simultaneously, and/or following, administration of the additional therapeutic agent and/or adjuvant. The pair of binding molecules of the invention may also be used in combination with radiation therapy.

### Articles of Manufacture

In another aspect of the invention, an article of manufacture containing materials useful for the treatment, prevention and/or diagnosis of the disorders described above (e.g. cancer) is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). In some aspects, the composition comprises the pair of binding molecules of the invention. The article of manufacture in these aspects may further comprise a label or package insert indicating that the composition can be used to treat a particular condition (e.g. cancer). In other aspects, the article of manufacture comprises (a) a first container with a composition contained therein, wherein the composition comprises the first binding molecule; and (b) a second container with a composition contained therein, wherein the composition comprises the second binding molecule. The article of manufacture in these aspects may further comprise a package insert indicating that the compositions can be used in combination to treat a particular condition (e.g. cancer).

In some aspects, the invention provides an article of manufacture (kit) intended for the treatment of a disease (e.g. cancer), comprising (i) a container comprising a pharmaceutical composition, wherein the pharmaceutical composition comprises the pair of binding molecules of the invention and an optional pharmaceutically acceptable carrier, and optionally (ii) a label or package insert containing instructions for using the pharmaceutical composition in the treatment of the disease (e.g. cancer).

In other aspects, the invention provides an article of manufacture (kit) intended for the treatment of a disease (e.g. cancer), comprising (i) a first container comprising a first pharmaceutical composition, wherein the first pharmaceutical composition comprises the first binding molecule of the pair of binding molecules of the invention and an optional pharmaceutically acceptable carrier, (ii) a second container comprising a second pharmaceutical composition, wherein the second pharmaceutical composition comprises the second binding molecule of the pair of binding molecules of the invention and an optional pharmaceutically acceptable carrier, and optionally (iii) a label or package insert containing instructions for using the first and the second pharmaceutical composition in combination in the treatment of the disease (e.g. cancer).

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Method for Forming a Functional Effector Domain

In a further aspect, the invention provides a method for forming a functional effector domain, comprising contacting the pair of binding molecules of the invention with a cell expressing the target antigens of the first and the second antigen binding domains, under conditions allowing binding of the first and the second antigen binding domain to their target antigens on the surface of the cell.

Amino Acid Sequences

| | | SEQ |
|---|---|---|
| | Sequence | ID NO |
| HER2 | EVQLVESGGGLVQPGGSLRLSCAASGFNIKDTYIHWVRQA | 1 |
| HC(knob) | PGKGLEWVARIYPTNGYTRYADSVKGRFTISADTSKNTAY | |
| | LQMNSLRAEDTAVYYCSRWGGEGFYAMDYWGQGTLVTV | |
| | SSASTKGPSVFPLAPSSKSTSGGTAALGCLVKDYFPEPVTVS | |
| | WNSGALTSGVHTFPAVLQSSGLYSLSSVVTVPSSSLGTQTY | |
| | ICNVNHKPSNTKVDKKVEPKSCDKTHTCPPCPAPEAAGGPS | |
| | VFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVYTLPPCRDELT | |
| | KNQVSLWCLVKGFYPSDIAVEWESNGQPENNYKTTPPVLD | |
| | SDGSFFLYSKLTVDKSRWQQGNVFSCSVMHEALHNHYTQ | |
| | KSLSLSP | |
| HER2 LC | DIQMTQSPSSLSASVGDRVTITCKASQDVSTAVAWYQQKP | 2 |
| | GKAPKLLIYSASFRYTGVPSRFSGSRSGTDFTLTISSLQPEDF | |
| | ATYYCQQHYTTPPTFGQGTKVEIKRTVAAPSVFIFPPSDEQL | |
| | KSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| HC(hole)- | DKTHTCPPCPAPEAAGGPSVFLFPPKPKDTLMISRTPEVTCV | 3 |
| CD3 VH (V9) | VVDVSHEDPEVKFNWYVDGVEVHNAKTKPREEQYNSTYR | |
| | VVSVLTVLHQDWLNGKEYKCKVSNKALGAPIEKTISKAKG | |
| | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSEVQLV | |
| | ESGGGLVQPGGSLRLSCAASGYSFTGYTMNWVRQAPGKG | |
| | LEWVALINPYKGVSTYNQKFKDRFTISVDKSKNTAYLQMN | |
| | SLRAEDTAVYYCARSGYYGDSDWYFDVWGQGTLVTVSS | |
| HC(hole)- | DKTHTCPPCPAPEAAGGPSVFLFPPKPKDTLMISRTPEVTCV | 4 |
| CD3 VH (V9)- | VVDVSHEDPEVKFNWYVDGVEVHNAKTKPREEQYNSTYR | |
| DP47 VL | VVSVLTVLHQDWLNGKEYKCKVSNKALGAPIEKTISKAKG | |
| QK | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSEVQLV | |
| | ESGGGLVQPGGSLRLSCAASGYSFTGYTMNWVRQAPGKG | |
| | LEWVALINPYKGVSTYNQKFKDRFTISVDKSKNTAYLQMN | |
| | SLRAEDTAVYYCARSGYYGDSDWYFDVWGQGTLVTVSSG | |
| | GGGSGGGGSGGGGSGGSGGEIVLTQSPGTLSLSPGERATLS | |
| | CRASQSVSSSYLAWYQKKPGQAPRLLIYGASSRATGIPDRF | |
| | SGSGSGTDFTLTISRLEPEDFAVYYCQQYGSSPLTFGQGTK | |
| | VEIK | |
| HC(hole)- | DKTHTCPPCPAPEAAGGPSVFLFPPKPKDTLMISRTPEVTCV | 5 |
| CD3 VL (V9) | VVDVSHEDPEVKFNWYVDGVEVHNAKTKPREEQYNSTYR | |
| | VVSVLTVLHQDWLNGKEYKCKVSNKALGAPIEKTISKAKG | |
| | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSDIQMT | |
| | QSPSSLSASVGDRVTITCRASQDIRNYLNWYQQKPGKAPKL | |
| | LIYYTSRLESGVPSRFSGSGSGTDYTLTISSLQPEDFATYYC | |
| | QQGNTLPWTFGQGTKVEIK | |
| HC(hole)- | DKTHTCPPCPAPEAAGGPSVFLFPPKPKDTLMISRTPEVTCV | 6 |
| CD3 VL (V9)- | VVDVSHEDPEVKFNWYVDGVEVHNAKTKPREEQYNSTYR | |
| DP47 VH | VVSVLTVLHQDWLNGKEYKCKVSNKALGAPIEKTISKAKG | |
| QE | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSDIQMT | |
| | QSPSSLSASVGDRVTITCRASQDIRNYLNWYQQKPGKAPKL | |
| | LIYYTSRLESGVPSRFSGSGSGTDYTLTISSLQPEDFATYYC | |
| | QQGNTLPWTFGQGTKVEIKGGGGSGGGGSGGGGSGGSGG | |
| | EVQLLESGGGLVQPGGSLRLSCAASGFTFSSYAMSWVREA | |
| | PGKGLEWVSAISGSGGSTYYADSVKGRFTISRDNSKNTLYL | |
| | QMNSLRAEDTAVYYCAKGSGFDYWGQGTLVTVSSAST | |
| HC(hole)- | DKTHTCPPCPAPEAAGGPSVFLFPPKPKDTLMISRTPEVTCV | 7 |
| CD3 VH | VVDVSHEDPEVKFNWYVDGVEVHNAKTKPREEQYNSTYR | |
| (P035.093) | VVSVLTVLHQDWLNGKEYKCKVSNKALGAPIEKTISKAKG | |
| | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSEVQLL | |
| | ESGGGLVQPGGSLRLSCAASGFTFSSYAMNWVRQAPGKGL | |
| | EWVSRIRSKYNNYATYYADSVKGRFTISRDDSKNTLYLQM | |
| | NSLRAEDTAVYYCVRASNFPASYVSYFAYWGQGTLVTVSS | |
| HC(hole)- | DKTHTCPPCPAPEAAGGPSVFLFPPKPKDTLMISRTPEVTCV | 8 |
| CD3 VH | VVDVSHEDPEVKFNWYVDGVEVHNAKTKPREEQYNSTYR | |
| (P035.093)- | VVSVLTVLHQDWLNGKEYKCKVSNKALGAPIEKTISKAKG | |
| DP47 VL QK | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSEVQLL | |
| | ESGGGLVQPGGSLRLSCAASGFTFSSYAMNWVRQAPGKGL | |
| | EWVSRIRSKYNNYATYYADSVKGRFTISRDDSKNTLYLQM | |
| | NSLRAEDTAVYYCVRASNFPASYVSYFAYWGQGTLVTVSS | |
| | GGGGSGGGGSGGGGSGGSGGEIVLTQSPGTLSLSPGERATL | |
| | SCRASQSVSSSYLAWYQKKPGQAPRLLIYGASSRATGIPDR | |
| | FSGSGSGTDFTLTISRLEPEDFAVYYCQQYGSSPLTFGQGTK | |
| | VEIK | |
| HC(hole)- | DKTHTCPPCPAPEAAGGPSVFLFPPKPKDTLMISRTPEVTCV | 9 |
| CD3 VL | VVDVSHEDPEVKFNWYVDGVEVHNAKTKPREEQYNSTYR | |
| (P035.093) | VVSVLTVLHQDWLNGKEYKCKVSNKALGAPIEKTISKAKG | |
| | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVL | |
| HC(hole)- | DKTHTCPPCPAPEAAGGPSVFLFPPKPKDTLMISRTPEVTCV | 10 |
| CD3 VL | VVDVSHEDPEVKFNWYVDGVEVHNAKTKPREEQYNSTYR | |
| (P035.093)- | VVSVLTVLHQDWLNGKEYKCKVSNKALGAPIEKTISKAKG | |
| DP47 VH QE | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSGGGGSGGGGSGGGG | |
| | SGGSGGEVQLLESGGGLVQPGGSLRLSCAASGFTFSSYAMS | |
| | WVREAPGKGLEWVSAISGSGGSTYYADSVKGRFTISRDNS | |
| | KNTLYLQMNSLRAEDTAVYYCAKGSGFDYWGQGTLVTVS | |
| | SAST | |
| HC(hole)- | DKTHTCPPCPAPEAAGGPSVFLFPPKPKDTLMISRTPEVTCV | 11 |
| CD3 VH | VVDVSHEDPEVKFNWYVDGVEVHNAKTKPREEQYNSTYR | |
| (40G5c) | VVSVLTVLHQDWLNGKEYKCKVSNKALGAPIEKTISKAKG | |
| | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSEVQLV | |
| | QSGAEVKKPGASVKVSCKASGYTFTNYYIHWVRQAPGQG | |
| | LEWIGWIYPGDGNTKYNEKFKGRATLTADTSTSTAYLELSS | |
| | LRSEDTAVYYCARDSYSNYYFDYWGQGTLVTVSS | |
| HC(hole)- | DKTHTCPPCPAPEAAGGPSVFLFPPKPKDTLMISRTPEVTCV | 12 |
| CD3 VH | VVDVSHEDPEVKFNWYVDGVEVHNAKTKPREEQYNSTYR | |
| (40G5c)- | VVSVLTVLHQDWLNGKEYKCKVSNKALGAPIEKTISKAKG | |
| DP47 VL QK | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSEVQLV | |
| | QSGAEVKKPGASVKVSCKASGYTFTNYYIHWVRQAPGQG | |
| | LEWIGWIYPGDGNTKYNEKFKGRATLTADTSTSTAYLELSS | |
| | LRSEDTAVYYCARDSYSNYYFDYWGQGTLVTVSSGGGGS | |
| | GGGGSGGGGSGGSGGEIVLTQSPGTLSLSPGERATLSCRAS | |
| | QSVSSSYLAWYQKKPGQAPRLLIYGASSRATGIPDRESGSG | |
| | SGTDFTLTISRLEPEDFAVYYCQQYGSSPLTFGQGTKVEIK | |
| HC(hole)- | DKTHTCPPCPAPEAAGGPSVFLFPPKPKDTLMISRTPEVTCV | 13 |
| CD3 VL | VVDVSHEDPEVKFNWYVDGVEVHNAKTKPREEQYNSTYR | |
| (40G5c) | VVSVLTVLHQDWLNGKEYKCKVSNKALGAPIEKTISKAKG | |
| | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSDIVMT | |
| | QSPDSLAVSLGERATINCKSSQSLLNSRTRKNYLAWYQQKP | |
| | GQPPKLLIYWASTRESGVPDRFSGSGSGTDFTLTISSLQAED | |
| | VAVYYCTQSFILRTFGQGTKVEIK | |
| HC(hole)- | DKTHTCPPCPAPEAAGGPSVFLFPPKPKDTLMISRTPEVTCV | 14 |
| CD3 VL | VVDVSHEDPEVKFNWYVDGVEVHNAKTKPREEQYNSTYR | |
| (40G5c)- | VVSVLTVLHQDWLNGKEYKCKVSNKALGAPIEKTISKAKG | |
| DP47 VH QE | QPREPQVCTLPPSRDELTKNQVSLSCAVKGFYPSDIAVEWE | |
| | SNGQPENNYKTTPPVLDSDGSFFLVSKLTVDKSRWQQGNV | |
| | FSCSVMHEALHNHYTQKSLSLSPGKGGGGSGGGGSDIVMT | |
| | QSPDSLAVSLGERATINCKSSQSLLNSRTRKNYLAWYQQKP | |
| | GQPPKLLIYWASTRESGVPDRFSGSGSGTDFTLTISSLQAED | |
| | VAVYYCTQSFILRTFGQGTKVEIKGGGGSGGGGSGGGGSG | |
| | GSGGEVQLLESGGGLVQPGGSLRLSCAASGFTFSSYAMSW | |
| | VREAPGKGLEWVSAISGSGGSTYYADSVKGRFTISRDNSKN | |
| | TLYLQMNSLRAEDTAVYYCAKGSGFDYWGQGTLVTVSSA | |
| | ST | |
| V9 HCDR1 | GYTMN | 15 |
| V9 HCDR2 | LINPYKGVSTYNQKFKD | 16 |
| V9 HCDR3 | SGYYGDSDWYFDV | 17 |
| V9 VH | EVQLVESGGGLVQPGGSLRLSCAASGYSFTGYTMNWVRQ | 18 |
| | APGKGLEWVALINPYKGVSTYNQKFKDRFTISVDKSKNTA | |
| | YLQMNSLRAEDTAVYYCARSGYYGDSDWYFDVWGQGTL | |
| | VTVSS | |
| V9 LCDR1 | RASQDIRNYLN | 19 |
| V9 LCDR2 | YTSRLES | 20 |
| V9 LCDR3 | QQGNTLPWT | 21 |
| V9 VL | DIQMTQSPSSLSASVGDRVTITCRASQDIRNYLNWYQQKPG | 22 |
| | KAPKLLIYYTSRLESGVPSRFSGSGSGTDYTLTISSLQPEDFA | |
| | TYYCQQGNTLPWTFGQGTKVEIK | |
| P035.093 | SYAMN | 23 |
| HCDR1 | | |
| P035.093 | RIRSKYNNYATYYADSVKG | 24 |
| HCDR2 | | |
| P035.093 | ASNFPASYVSYFAY | 25 |
| HCDR3 | | |
| P035.093 VH | EVQLLESGGGLVQPGGSLRLSCAASGFTFSSYAMNWVRQA | 26 |
| | PGKGLEWVSRIRSKYNNYATYYADSVKGRFTISRDDSKNT | |
| | LYLQMNSLRAEDTAVYYCVRASNFPASYVSYFAYWGQGT | |
| | LVTVSS | |
| P035.093 | GSSTGAVTTSNYAN | 27 |
| LCDR1 | | |
| P035.093 | GTNKRAP | 28 |
| LCDR2 | | |
| P035.093 | ALWYSNLWV | 29 |
| LCDR3 | | |
| P035.093 VL | QAVVTQEPSLTVSPGGTVTLTCGSSTGAVTTSNYANWVQE | 30 |
| | KPGQAFRGLIGGTNKRAPGTPARFSGSLLGGKAALTLSGAQ | |
| | PEDEAEYYCALWYSNLWVFGGGTKLTVL | |
| 40G5c | NYYIH | 31 |
| HCDR1 | | |
| 40G5c | WIYPGDGNTKYNEKFKG | 32 |
| HCDR2 | | |
| 40G5c | DSYSNYYFDY | 33 |
| HCDR3 | | |
| 40G5c VH | EVQLVQSGAEVKKPGASVKVSCKASGYTFTNYYIHWVRQ | 34 |
| | APGQGLEWIGWIYPGDGNTKYNEKFKGRATLTADTSTSTA | |
| | YLELSSLRSEDTAVYYCARDSYSNYYFDYWGQGTLVTVSS | |
| 40G5c | KSSQSLLNSRTRKNYLA | 35 |
| LCDR1 | | |
| 40G5c | WASTRES | 36 |
| LCDR2 | | |
| 40G5c | TQSFILRT | 37 |
| LCDR3 | | |
| 40G5c VL | DIVMTQSPDSLAVSLGERATINCKSSQSLLNSRTRKNYLAW | 38 |
| | YQQKPGQPPKLLIYWASTRESGVPDRFSGSGSGTDFTLTISS | |
| | LQAEDVAVYYCTQSFILRTFGQGTKVEIK | |
| HER2 HCDR1 | DTYIH | 39 |
| HER2 HCDR2 | RIYPTNGYTRYADSVKG | 40 |
| HER2 HCDR3 | WGGEGFYAMDY | 41 |
| HER2 VH | EVQLVESGGGLVQPGGSLRLSCAASGFNIKDTYIHWVRQA | 42 |
| | PGKGLEWVARIYPTNGYTRYADSVKGRFTISADTSKNTAY | |
| | LQMNSLRAEDTAVYYCSRWGGEGFYAMDYWGQGTLVTV | |
| | SS | |
| HER2 LCDR1 | KASQDVSTAVA | 43 |
| HER2 LCDR2 | SASFRYT | 44 |
| HER2 LCDR3 | QQHYTTPPT | 45 |
| HER2 VL | DIQMTQSPSSLSASVGDRVTITCKASQDVSTAVAWYQQKP | 46 |
| | GKAPKLLIYSASFRYTGVPSRFSGSRSGTDFTLTISSLQPEDF | |
| | ATYYCQQHYTTPPTFGQGTKVEIK | |
| DP47 HCDR1 | SYAMS | 47 |
| DP47 HCDR2 | AISGSGGSTYYADSVKG | 48 |
| DP47 HCDR3 | GSGFDY | 49 |
| DP47 VH QE | EVQLLESGGGLVQPGGSLRLSCAASGFTFSSYAMSWVREA | 50 |
| | PGKGLEWVSAISGSGGSTYYADSVKGRFTISRDNSKNTLYL | |
| | QMNSLRAEDTAVYYCAKGSGFDYWGQGTLVTVSS | |
| DP47 LCDR1 | RASQSVSSSYLA | 51 |
| DP47 LCDR2 | GASSRAT | 52 |
| DP47 LCDR3 | QQYGSSPLT | 53 |
| DP47 VL QK | EIVLTQSPGTLSLSPGERATLSCRASQSVSSSYLAWYQKKP | 54 |
| | GQAPRLLIYGASSRATGIPDRESGSGSGTDFTLTISRLEPEDF | |
| | AVYYCQQYGSSPLTFGQGTKVEIK | |
| Linker | GGGGSGGGGS | 55 |
| Linker | GGGGSGGGGSGGGGSGGSGG | 56 |
| Human CD3E | QDGNEEMGGITQTPYKVSISGTTVILTCPQYPGSEILWQHN | 57 |
| | DKNIGGDEDDKNIGSDEDHLSLKEFSELEQSGYYVCYPRGS | |
| | KPEDANFYLYLRARVCENCMEMDVMSVATIVIVDICITGG | |
| | LLLLVYYWSKNRKAKAKPVTRGAGAGGRQRGQNKERPPP | |
| | VPNPDYEPIRKGQRDLYSGLNQRRI | |
| Cynomolgus | QDGNEEMGSITQTPYQVSISGTTVILTCSQHLGSEAQWQHN | 58 |
| CD3E | GKNKEDSGDRLFLPEFSEMEQSGYYVCYPRGSNPEDASHH | |
| | LYLKARVCENCMEMDVMAVATIVIVDICITLGLLLLVYYW | |
| | SKNRKAKAKPVTRGAGAGGRQRGQNKERPPPVPNPDYEPI | |
| | RKGQQDLYSGLNQRRI | |
| Human IgG1 | DKTHTCPPCPAPELLGGPSVFLFPPKPKDTLMISRTPEVTCV | 59 |
| Fc | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |
| C22 HCDR3 | HTTFPSSYVSYYGY | 60 |
| C22 VH | EVQLLESGGGLVQPGGSLRLSCAASGFQFSSYAMNWVRQA | 61 |
| | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSS | |

### BRIEF DESCRIPTION OF THE DRAWINGS

FIG. 1. Schematic illustration of exemplary SPLIT molecules according to the invention. (A) SPLIT molecule with bivalent binding to target antigen and non-complemented effector VL domain. (B) (A) SPLIT molecule with bivalent binding to target antigen and non-complemented effector VH domain. (C) SPLIT molecule with monovalent binding to target antigen and non-complemented effector VL domain. (D) SPLIT molecule with monovalent binding to target antigen and non-complemented effector VH domain. (E) SPLIT molecule with monovalent binding to target antigen and V-complemented effector VL domain. (F) SPLIT molecule with monovalent binding to target antigen and V-complemented effector VH domain. Circles in Fc region represent modifications to promote Fc heterodimerization such as “knob-into-hole” modifications.

FIG. 2. The molecules with different split anti-CD3 binders were tested in a binding assay with Jurkat cells as target cells. Binding on Jurkat cells was determined by measuring median fluorescence intensity (MFI) by flow cytometry as described in the methods. MFI results are shown for the V9 binder (A), for the P035.093 binder (B), for the 40G5c binder (C) and for the C22 binder (D), with the complemented molecules shown with gray circles and non-complemented molecules with black circles.
P1AF1375+P1AF1376: SPLIT molecule with non-complemented V9 binder.
P1AF3870+P1AF3871: SPLIT molecule with V-complemented V9 binder.
P1AF2484+P1AF2678: SPLIT molecule with non-complemented P035.093 binder.
P1AF3868+P1AF3869: SPLIT molecule with V-complemented P035.093 binder.
P1AA9518+P1AA9516: SPLIT molecule with non-complemented 40G5c binder.
P1AF3865+P1AF3867: SPLIT molecule with V-complemented 40G5c binder.
P1AF2678+P1AE6818: SPLIT molecule with non-complemented C22 binder.
The format of all molecules is analogous to the molecules shown in Table 2.

FIG. 3. Tumor cell killing of the ovarian adenocarcinoma cell line SKOV-3 (HER-2 positive) with CD3+ T cells from a healthy donor was assessed when treated with SPLIT molecules with different anti-CD3 binders. Tumor cell killing was measured by quantification of cell death using the CytotoxGlo kit (Promega) after 48 hours. Cell death results are shown for the V9 binder (A), for the P035.093 binder (B), for the 40G5c binder (C). Pairs are represented with filled symbols and single prodrugs as open symbols.
P1AD4471: Control molecule (non-SPLIT HER2×CD3 bispecific antibody (with HER2 bivalent and CD3 monovalent binding)).
P1AF3870+P1AF3871: HER2-targeted SPLIT molecule with complemented V9 binder.
P1AF3868+P1AF3869: HER2-targeted SPLIT molecule with complemented P035.093 binder.
P1AE6814+P1AF2484: HER2-targeted SPLIT molecule with non-complemented P035.093 binder.
P1AF3865+P1AF3867: HER2-targeted SPLIT molecule with complemented 40G5c binder.
P1AD9224+P1AD9225: HER2-targeted SPLIT molecule with non-complemented 40G5c binder (having charged residues in the VH (Q39E) and VL (Q38K) domains, as described hereinabove).
The format of all SPLIT molecules is analogous to the molecules shown in Table 2.

FIG. 4. Tumor cell killing of the ovarian adenocarcinoma cell line SKOV-3 (HER-2 positive) with CD3+ T cells from a healthy donor was assessed when treated with SPLIT molecules containing the anti-CD3 binder P035.093. Tumor cell killing was measured by quantification of cell death using the CytotoxGlo kit (Promega) after 48 hours. Cell death results are shown for the non-complemented (A) and complemented (B) molecules. Positive control molecule (non-SPLIT HER2×CD3 bispecific antibody) is shown as filled squares, HER2+HER2 productive pair represented by filled circles, while HER2+irrelevant binder is shown with open symbols.
P1AD4471: Control molecule (non-SPLIT HER2×CD3 bispecific antibody).
P1AE6814+P1AF2484: HER2-targeted SPLIT molecule with non-complemented P035.093 binder.
P1AF2484+P1AG7469: SPLIT molecule with one HER2-targeted prodrug and one untargeted (DP47) prodrug, with non-complemented P035.093 binder.
P1AF3868+P1AF3869: HER2-targeted SPLIT molecule with complemented P035.093 binder.
P1AF3869+P1AG5786: SPLIT molecule with one HER2-targeted prodrug and one untargeted (DP47) prodrug, with complemented P035.093 binder.
The format of all SPLIT molecules is analogous to the molecules shown in Table 2.

FIG. 5. Tumor cell killing of the ovarian adenocarcinoma cell line SKOV-3 (HER-2 positive) with CD3+ T cells from a healthy donor was assessed. Tumor cell killing was measured by quantification of cell death using the CytotoxGlo kit (Promega) after 48 hours. Cell death results are shown for the non-complemented molecules containing the 40G5c binder (A) and the C22 binder (B). Positive control molecule (non-SPLIT HER2×CD3 bispecific antibody) is shown as filled squares, HER2+HER2 productive pair represented by filled circles, while HER2+irrelevant binder is shown with open symbols.
P1AD4471: Control molecule (non-SPLIT HER2×CD3 bispecific antibody).
P1AA9516+P1AA9518: HER2-targeted SPLIT molecule with non-complemented 40G5c binder.
P1AA9518+P1AG7808: SPLIT molecule with one HER2-targeted prodrug and one untargeted (DP47) prodrug, with non-complemented 40G5c binder.
P1AE6814+P1AE6818: HER2-targeted SPLIT molecule with non-complemented C22 binder.
P1AE6818+P1AG7469: SPLIT molecule with one HER2-targeted prodrug and one untargeted (DP47) prodrug, with non-complemented C22 binder.
The format of all SPLIT molecules is analogous to the molecules shown in Table 2.

FIG. 6. Tumor cell killing of the ovarian adenocarcinoma cell line SKOV-3 (HER-2 high expressor) and the prostate carcinoma cell line LNCaP (HER-2 low expressor) with CD3+ T cells from a healthy donor was assessed. Tumor cell killing was measured by quantification of cell death using the CytotoxGlo kit (Promega) after 48 hours. Cell death results are shown for the SKOV-3 cell line (A) and for the LNCaP cell line (B), together with their respective antigen binding sites quantification (C). The HER2+HER2 productive pair is represented by filled circles, while the HER2+irrelevant binder pair is shown with open symbols.
P1AF3868+P1AF3869: HER2-targeted SPLIT molecule with complemented P035.093 binder.
P1AF3869+P1AG5786: SPLIT molecule with one HER2-targeted prodrug and one untargeted (DP47) prodrug, with non-complemented P35.093 binder.
The format of all molecules is analogous to the molecules shown in Table 2.

FIG. 7. Tumor cell killing of the ovarian adenocarcinoma cell line SKOV-3 (HER-2 high expressor) and the prostate carcinoma cell line LNCaP (HER-2 low expressor) with CD3+ T cells from a healthy donor was assessed. Tumor cell killing was measured by quantification of cell death using the CytotoxGlo kit (Promega) after 48 hours. Cell death results are shown for the SKOV-3 cell line (A) and for the LNCaP cell line (B). The non-complemented productive pairs for the various CD3 binders are shown as 40G5c (squares), C22 (circles), P035.093 (diamonds). P1AA9518+P1AA9516: HER2-targeted SPLIT molecule with non-complemented 40G5c binder.
P1AE6818+P1AE6814: HER2-targeted SPLIT molecule with non-complemented C22 binder.
P1AF2484+P1AE6814: HER2-targeted SPLIT molecule with non-complemented P035.093 binder.
The format of all molecules is analogous to the molecules shown in Table 2.

FIG. 8. Ex vivo analysis of human T cells from humanized mice. Mice were injected with equimolar doses of SPLIT molecules and blood was collected and analyzed 24 hours post-treatment to assess target-independent CD3 binder assembly in circulation. T cell bound molecules were measured by flow cytometry using a PE-labeled anti-human IgG, Fc-specific secondary antibody. Median fluorescence intensity is shown for CD4+ (A) and CD8+ T cells (B) from blood of treated animals with non-complemented and complemented P035.093 compounds.
P1AE6814+P1AF2484: HER2-targeted SPLIT molecule with non-complemented P035.093 binder.
P1AF3868+P1AF3869: HER2-targeted SPLIT molecule with complemented P035.093 binder.
The format of all molecules is analogous to the molecules shown in Table 2.

FIG. 9. Ex vivo analysis of human T cells from humanized mice. Mice were injected with equimolar doses of SPLIT molecules and blood was collected and analyzed 24 hours post-treatment to assess target-independent CD3 binder assembly in circulation. T cell bound molecules were measured by flow cytometry using a PE-labeled anti-human IgG, Fc-specific secondary antibody. Median fluorescence intensity is shown for CD4+ (A) and CD8+ T cells (B) from blood of treated animals with non-complemented and complemented V9 compounds.
P1AF1375+P1AF1376: tumor antigen-targeted SPLIT molecule with non-complemented V9 binder.
P1AF3870+P1AF3871: HER2-targeted SPLIT molecule with complemented V9 binder.
The format of all molecules is analogous to the molecules shown in Table 2.

FIG. 10. Ex vivo analysis of human T cells from humanized mice. Mice were injected with equimolar doses of SPLIT molecules and blood was collected and analyzed 24 hours post-treatment to assess target-independent CD3 binder assembly in circulation. T cell bound molecules were measured by flow cytometry using a PE-labeled anti-human IgG, Fc-specific secondary antibody. Median fluorescence intensity is shown for CD4+ (A) and CD8+ T cells (B) from blood of treated animals with non-complemented compounds based on C22 and 40G5c as CD3 binders.
P1AF6814+P1AF6818: HER2-targeted SPLIT molecule with non-complemented C22 binder.
P1AF9516+P1AF9518: HER2-targeted SPLIT molecule with non-complemented 40G5c binder.
The format of all molecules is analogous to the molecules shown in Table 2.

### EXAMPLES

The following are examples of methods and compositions of the invention. It is understood that various other aspects may be practiced, given the general description provided above.

### Example 1. Generation of SPLIT Molecules with or without V-Complementation

### 1.1 Construction of the Expression Plasmids for the SPLIT Molecules

For the expression of the SPLIT constructs as reported herein a transcription unit comprising the following functional elements was used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a nucleic acid encoding the respective fusion polypeptide, and
the bovine growth hormone polyadenylation sequence (BGH pA).
   Beside the expression unit/cassette including the desired gene, the basic/standard mammalian expression plasmid contains
an origin of replication from the vector pUC18 which allows replication of this plasmid in E. coli, and
a beta-lactamase gene which confers ampicillin resistance in E. coli.

### 1.2 Expression of the SPLIT Molecules

Transient expression of the SPLIT molecules was performed in suspension-adapted HEK293F (FreeStyle 293-F cells; Invitrogen) cells or Expi293 (Expi293F™ cells; Thermofisher Scientific) with Transfection Reagent 293-free (Novagen) or ExpiFectamine™ 293 Transfection Kit (ExpiFectamine™ 293 Reagent, ExpiFectamine™ 293 Transfection Enhancers 1 und 2).

Cells were passaged, by dilution, at least four times (volume 30 ml) after thawing in a 125 ml shake flask (incubate/shake at 37° C., 7% CO₂, 85% humidity, 135 rpm). The cells were expanded to 3×10⁵ cells/ml in 250 ml volume. Three days later, cells were split and newly seeded with a density of 1.5×10⁶ to 7×10⁵ cells/ml in a 250 ml volume in a 1 L shake flask. Transfection was performed 24 hours later at a cell density around 1.4-3.0×10⁶ cells/ml.

Before transfection 250 μg plasmid-DNA were diluted in a final volume of 10 ml with pre-heating (water bath; 37° C.) Opti-MEM (Gibco). The solution was gently mixed and incubated at room temperature for up to 5 min. For transfections with ExpiFectamine™. 675 μl of the ExpiFcctaminc™ 293 reagent were added to 12.5 ml OptiMEM-solution and incubated in a separate tube also for 5 minutes. Then the two solutions were united, mixed gently, and incubated at room temperature for 15-20 minutes, and the complete mixture was added to 1 L shake flask with 250 ml Expi293 cell culture. For transfections with 293-free, 333.3 μl of the 293-free transfection reagent were added to the DNA-OptiMEM-solution directly. The resulting solution was gently mixed and incubated at room temperature for 15-20 minutes. The complete volume of mixture was added to 1 L shake flask with 250 ml HEK293F cell culture.

The incubation was performed by shaking the flask at 37° C., 7% CO₂, 85% humidity, 135 rpm for 6 to 7 days.

The supernatant was harvested by a first centrifugation step at 2,000 rpm, 4° C., for 10 minutes. Then the supernatant was transferred into a new centrifugation flask for a second centrifugation at 4,000 rpm, 4° C., for 20 minutes. Thereafter, the cell-free supernatant was filtered through a 0.22 μm bottle top filter and stored in a freezer (−20° C.).

### 1.3 Purification of the SPLIT Molecules

The SPLIT molecule containing culture supernatants were filtered and purified by two chromatographic steps. The antibodies were captured by affinity chromatography using HiTrap MabSelectSuRe (GE Healthcare) equilibrated with PBS (1 mM KH₂PO₄, 10 mM Na₂HPO₄, 137 mM NaCl, 2.7 mM KCl), pH 7.4. Unbound proteins were removed by washing with equilibration buffer, and the SPLIT molecule was recovered with 50 mM citrate buffer, pH 2.8, and immediately after elution neutralized to pH 6.0 with 1 M Tris-base, pH 9.0. Alternatively, the SPLIT molecules were eluted from the MabSelectSuRe column with 100 mM acetic acid pH 3.0 and conditioned to pH 5.5. The protein sample recovered from the affinity chromatography was analyzed by analytical SEC, and dependent on the impurity profile, an additional purification step was carried out. As additional purification step, either an ion exchange column POROS XS or POROS HS50, or a hydroxyapatite column Macro Prep CHT Type I (BioRad), or a hydrophobic interaction chromatography (HIC) column TSkgel Ether-5 PW (Tosoh Bioscience, Griesheim) was employed. For the hydroxyapatite chromatography, the MabSelectSuRe eluate was either diluted with 10 mM sodium citrate, pH 5.0 (1:5 vol/vol) and the pH was adjusted to 7.5 with 2 M Tris/HCl, pH 9.0, or it was dialyzed to 50 mM acetate buffer pH 7.5. After adding a pinch of calcium chloride, the protein solution was loaded to the Macro Prep column equilibrated in 25 mM HEPES, 5 mM Na₂HPO₄, 50 mM NaCl, 0.1 mM CaCl₂), 100 mM MES, pH 6.8, and eluted with a gradient of the same buffer containing a final concentration of 1500 mM NaCl. On the POROS XS ion exchange chromatography, the SPLIT proteins were purified with a 20 mM His/His-HCl buffer at pH 5.5 with a salt gradient from 0 to 550 mM NaCl. Alternatively, VL fusion proteins were best prepared using a 40 mM Na-Acetate buffer with a combined pH/NaCl gradient from pH 4.5 to 5 and 125 mM to 600 mM NaCl. VH fusion proteins were best prepared on a POROS XS column in 20 mM Na-phosphate buffer pH 5.5 with a salt gradient from 5 to 500 mM NaCl. For the hydrophobic interaction chromatography a 40 mM sodium acetate buffer pH 5.5 was containing 1.5 M (NH₄)₂SO₄, and elution was achieved with a shallow gradient of 50 column volumes towards no salt content. Size exclusion chromatography on a Superdex 200™ column (GE Healthcare) was used as polishing step in all cases. The size exclusion chromatography was performed in 20 mM histidine buffer, 0.14 M NaCl, pH 6.0. Finally, the SPLIT molecule containing solutions were concentrated with an Ultrafree-CL centrifugal filter unit equipped with a Biomax-SK membrane (Millipore, Billerica, MA) and stored at −80° C.

### 1.4 Mass Spectrometric Analysis of the SPLIT Molecules

PNGase F was obtained from Roche Diagnostics GmbH (14.3 U/μl; solution in sodium phosphate, EDTA and glycerol). A protease specifically cleaving in the hinge region of an IgG antibody was freshly reconstituted from a lyophilisate prior to digestion.

Enzymatic deglycosylation of with PNGase F

50 μg of SPLIT molecule was diluted to a final concentration of 0.5 mg/ml with 10 mM sodium phosphate buffer, pH 7.1, and deglycosylated with 1 μl PNGase F at 37° C. for 16 hours.

### ESI-QTOF Mass Spectrometry

The digested samples were then analyzed by LC-MS. Liquid chromatography was performed on a Waters Acquity UPLC (Waters) with a reversed-phase C18 column (Agilent PLRP-S column, 2.1×150 mm, 8 μm, 1000 Å (Agilent, Cat.-Nr.: PL1912-3802)). The aqueous mobile phase (mobile phase A) contained 0.1% (v/v) formic acid (FA) in HPLC grade water. The organic mobile phase (mobile phase B) contained 0.1% FA in acetonitrile. The gradient that was utilized in this experiment is plotted in Table 1.

TABLE 1
Gradient used for LC-MS.

| | | % A1 | % B1 |
|---|---|---|---|
| Step (n) | Time (min) | (FA/water) | (FA/acetonitrile) |
| 1 | 0 | 90 | 10 |
| 2 | 1 | 75 | 25 |
| 3 | 16 | 55 | 45 |
| 4 | 17 | 5 | 95 |
| 5 | 19 | 5 | 95 |
| 6 | 19.5 | 90 | 10 |
| 7 | 21 | 90 | 10 |

Further chromatographic settings were as follows:
Flow rate: 0.6 mL/min
Temperature of the column oven: 75° C.
Injection volume: 8 μl

The UPLC was coupled to an ESI-QTOF MS instrument (maXis 4G UHR-QTOF MS system (Bruker Daltonik)). Calibration was performed with sodium iodide. For the digested SPLIT molecule, data acquisition was done at 800-4000 m/z (isCID: 85 eV). The raw mass spectra were evaluated and transformed into individual relative molar masses. For visualization of the results proprietary software was used to generate deconvoluted mass spectra.

The SPLIT molecules that were produced are shown in Table 2. The molecules did or did not comprise a complementing domain.

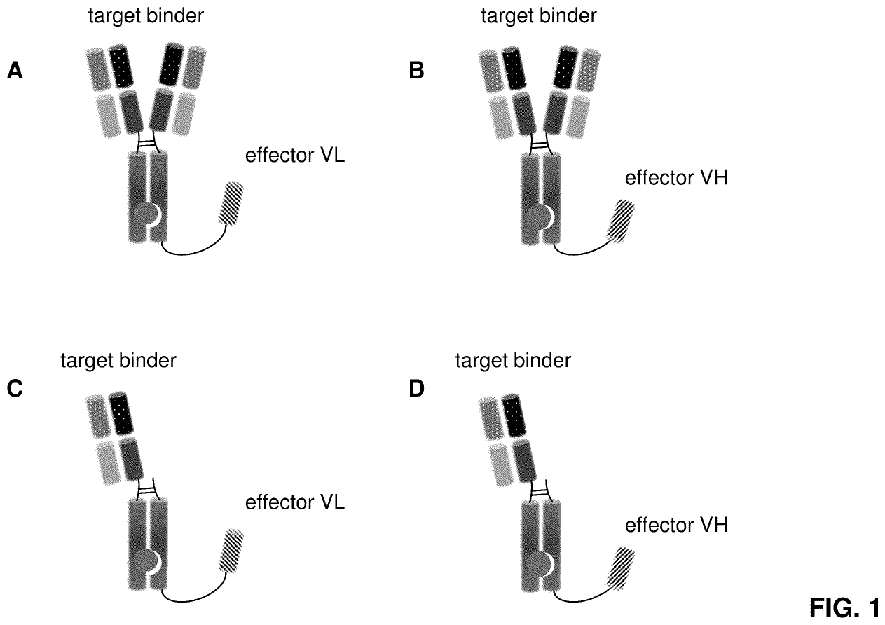

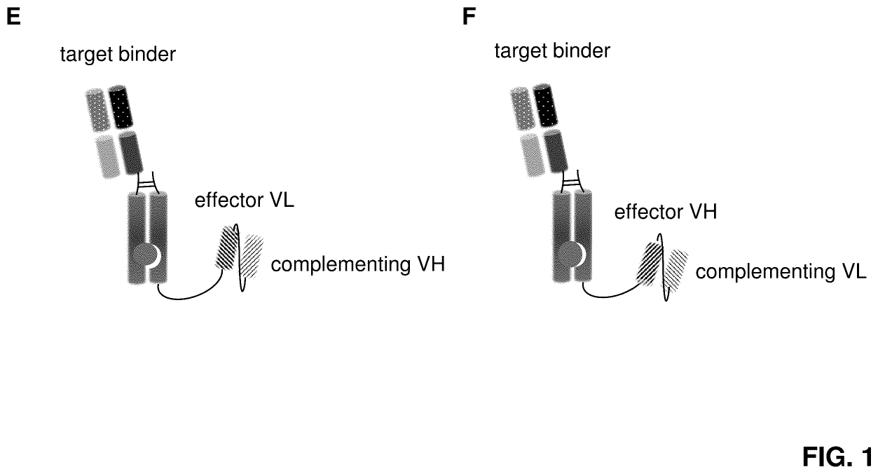

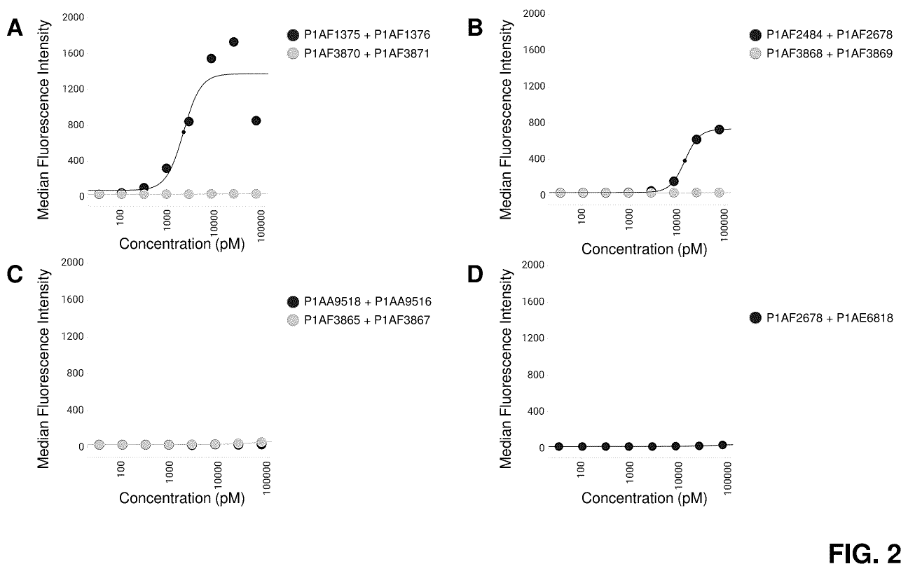

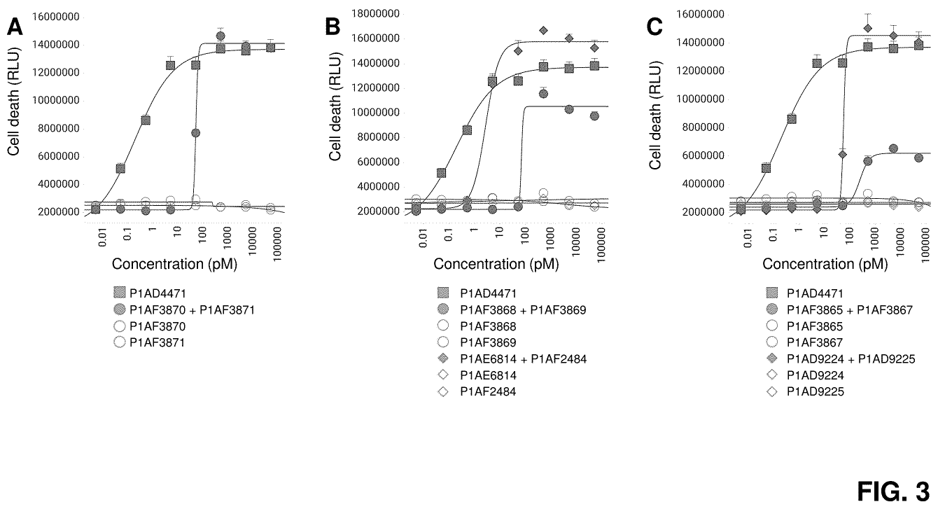

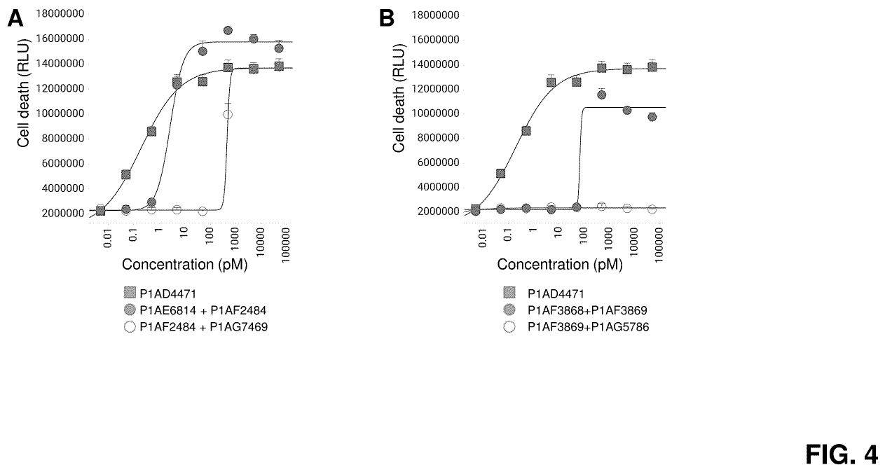

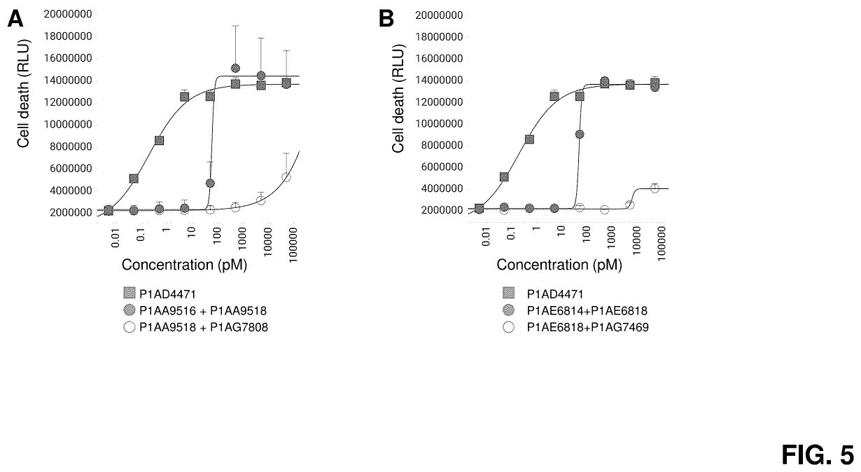

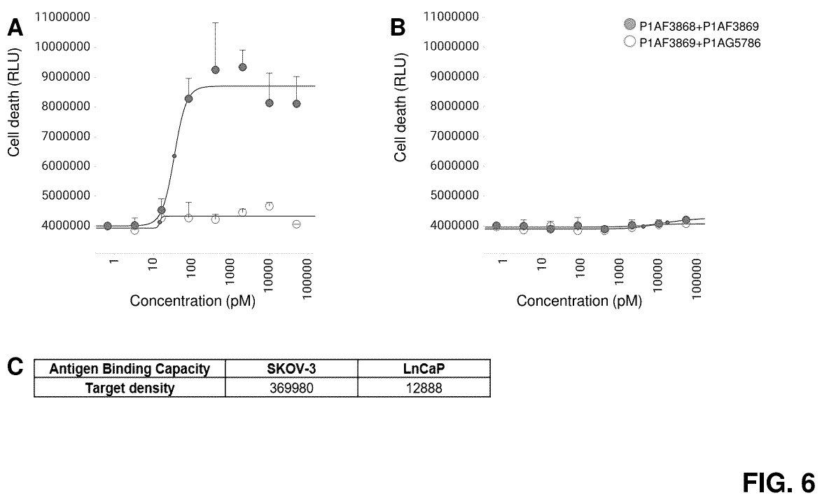

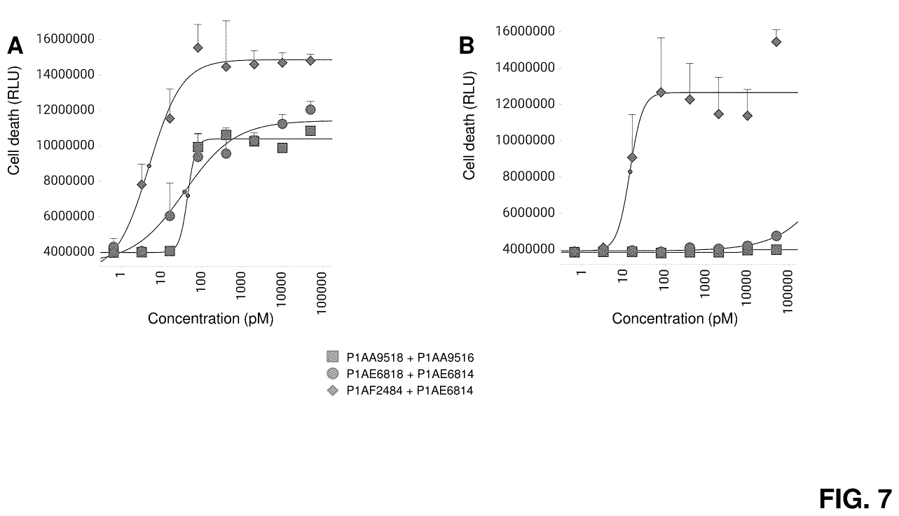

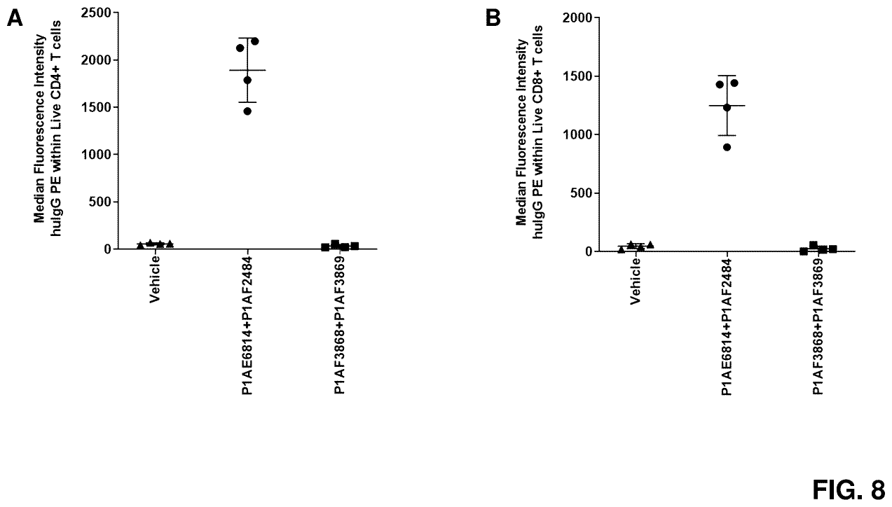

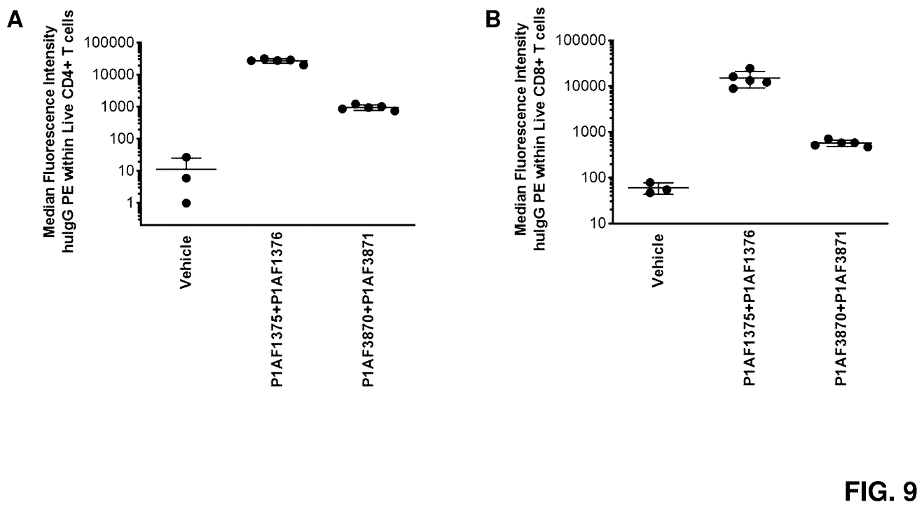

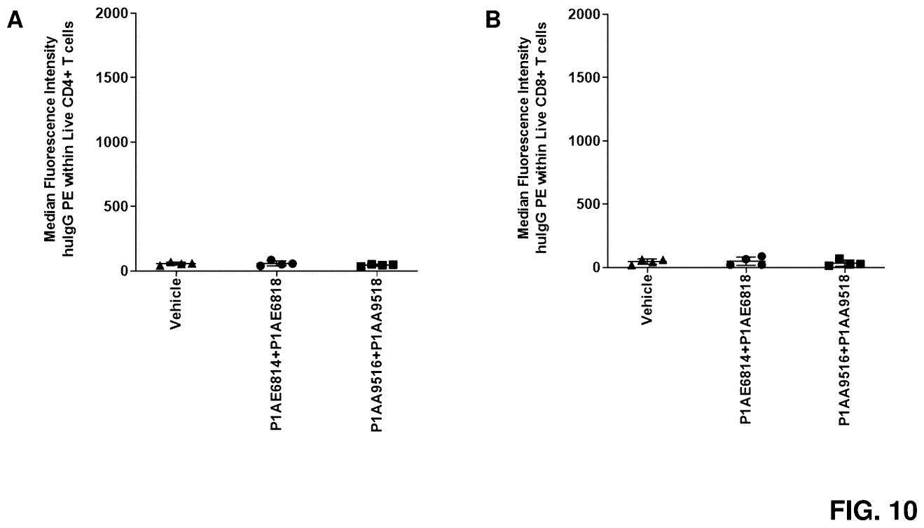

TABLE 2
SPLIT molecules with (shaded) or without (not shaded) V-complementation.

| | CD3 binder | Complementing | Target | Format | |
|---|---|---|---|---|---|
| Molecule | domain | domain | binder | FIG. 1 | Sequences |
| P1AE6819 | V9 VH | — | HER2 | D | 1, 2, 3 |
| P1AF3870 | V9 VH | DP47 VL | HER2 | F | 1, 2, 4 |
| P1AE6820 | V9 VL | — | HER2 | C | 1, 2, 5 |
| P1AF3871 | V9 VL | DP47 VH | HER2 | E | 1, 2, 6 |
| P1AF2484 | P035.093 VH | — | HER2 | D | 1, 2, 7 |
| P1AF3869 | P035.093 VH | DP47 VL | HER2 | F | 1, 2, 8 |
| P1AE6814 | P035.093 VL | — | HER2 | C | 1, 2, 9 |
| P1AF3868 | P035.093 VL | DP47 VH | HER2 | E | 1, 2, 10 |
| P1AA9518 | 40G5c VH | — | HER2 | D | 1, 2, 11 |
| P1AF3865 | 40G5c VH | DP47 VL | HER2 | F | 1, 2, 12 |
| P1AA9516 | 40G5c VL | — | HER2 | C | 1, 2, 13 |
| P1AF3867 | 40G5c VL | DP47 VH | HER2 | E | 1, 2, 14 |

The purification yield of the various SPLIT molecules is shown in Table 3.

For the CD3 binder V9, the complementation of the single VH and VL domains with the cognate DP47 V domains improved the production yield of the SPLIT constructs significantly. Especially the purification yield for the SPLIT VH construct of V9 was improved from 0.8 mg/L for the non-complemented SPLIT construct P1AE6819 to 20.8 mg/L for the DP47 complemented construct P1AF3870.

The V-complementation of the CD3 binder P035.093 with the cognate V domains of DP47 somewhat decreased the purification yields, but significantly increased the stability and developability of the SPLIT P035.093 constructs (see Example 2).

For 40G5c, V-complementation with DP47 greatly improved the purification procedure, as the non-complemented SPLIT constructs P1AA9518 and P1AA9516 could only be purified in sufficient yield via hydroxyapatite chromatography, while the V-complemented constructs P1AF3865 and P1AF3867 did not need this delicate purification method to obtain sufficient amounts.

### Example 2. Developability of SPLIT Molecules

### 2.1 Physicochemical Characterization

### Apparent Hydrophobicity

Apparent hydrophobicity was evaluated by determining the retention time on a hydrophobic HPLC column in comparison with known high and low hydrophobicity standard molecules.

### PK Prediction

Interaction with immobilized FcRn and Heparin on specific HPLC columns was evaluated. The corresponding retention times were compared to established thresholds based on known molecules with good and poor PK.

### Thermal Stability and Aggregation Propensity

Molecules were exposed to increasing temperature in a controlled gradient and aggregation propensity (T_{agg} by SLS) was determined.

The results of the apparent hydrophobicity, PK prediction, thermal stability and aggregration propensity are shown in Table 4 below.

### 2.2 Early Stability Assessment

### Storage

Molecules were stored under conditions mimicking both physiological and shelf life indicating conditions for prolonged periods of time at relevant temperatures (physiological and stressed conditions). Subsequent analysis focused on changes observed in the stored samples compared to an untreated control.

### Analysis

Aggregation of molecules was assessed using an SE-HPLC setup with conditions appropriate for the evaluated molecule. Fragmentation of molecules was assessed using capillary gel electrophoresis. Functional integrity of the molecules was assessed with a binding assay specific for the target(s) of the molecule.

The results of the early stability assessment are shown in Table 5 below.

Thermostability and stress resistance data of constructs with SPLIT CD3 domains of 40G5c met the acceptance criteria of the developability analysis for the non-complemented SPLIT constructs P1AA9518 and P1AA9516 already without the need of V-complementation.

V-complementation with DP47 of the P035.093 SPLIT constructs (P1AF3868 and P1AF3869) improved the developability properties significantly compared to the non-complemented SPLIT constructs P1AE6814 and P1AF2484, especially concerning the apparent hydrophobicity and the stress stability determined by SEC.

### Example 3. Functional Characterization of SPLIT Molecules

### 3.1 Methods

### Flow Cytometry Binding on Human T Cells

The binding of SPLIT molecules to human T cells was assessed. The molecule titrations in RPMI+10% FBS were added on the cells at an equimolar ratio for the complimentary VH/VL pairs of SPLIT molecules. Plates were incubated for 60 min at 4° C. in the dark. To remove non-bound molecules, plates were washed with cold PBS two times and cell pellets were resuspended in cold FACS buffer containing PE-conjugated anti-human Fc gamma-specific goat IgG F (ab), fragment (Jackson ImmunoResearch) as secondary detection antibody and Zombie Aqua (Biolegend) to identify living cells. Plates were incubated for 30 min at 4° C. in the dark, washed two times with cold PBS and resuspended in FACS buffer.

Cells were acquired using a FORTESSA flow cytometry device (Becton Dickinson) and an automated HTS plate-handling system.

Data was analyzed using FlowJo v10.8.1 for PC (FlowJo LLC), Microsoft Excel (Microsoft Office Standard 2016) and TIBCO SpotFire v10.10.4 (TIBCO Software Inc).

### T Cell Mediated Tumor Cell Killing

The molecules were tested in tumor cell killing assays with freshly isolated human CD3+ T cells, co-incubated with the target cells. Tumor cell lysis was determined by quantification of extracellular protease activity released into supernatants by apoptotic or necrotic cells as described below.

Target cells were detached using trypsin (Gibco), washed once with PBS and re-suspended at a density of 0.2 mio cells/ml in growth medium (RPMI 1640 (Gibco) containing 10% FBS, 1% GlutaMax (Gibco). 100 μl of the cell suspension (containing 20 000 cells) were seeded into a 96 well flat bottom plate and were incubated overnight at 37° C. in the incubator. CD3+ T cells were isolated from PBMCs after Ficoll isolation from blood of a healthy donor and viability was checked. Antibodies were diluted in assay medium at indicated concentrations and molecules were added to the target cells.

Assay medium was added to appropriate wells to reach equal volumes in each well. Isolated CD3+ T cells were resuspended at a density of 4 mio cells/ml, 50 μl were added per well resulting in 200 000 cells/well (E:T 10:1). For determination of spontaneous dead cell protease release, CD3+ effector cells and target cells were co-incubated as negative control.

The assay was incubated in total for 72 h at 37° C., 5% CO₂. Dead cell protease activity measurements were performed at 24, 48 and/or 72 hours after assay start as indicated in the figures. For this, the CytoTox-Glo™ Cytotoxicity Assay (Promega, #G9291) was adjusted to room temperature before measurement. 50 μl of supernatant per well was transferred to a 96 well white flat bottom plate for analysis. 25 μl of the substrate was subsequently added to each well and after minutes incubation at room temperature, luminescence was measured using a Perkin Elmer En Vision® 2104 instrument.

### Quantification of Antigen Density on Target Cells

Flow cytometric estimation of antibodies bound per cell was performed with BD Quantibrite™ Beads (Becton Dickinson #340495) and monoclonal PE-conjugated antibodies, targeting human CD340 (HER2) (Biolegend #324406). 200′000 cells of each cell line were harvested and washed twice with 200 μl FACS buffer. Diluted antibody was prepared with FACS buffer (1:100). Cells were centrifuged and resuspended in 100 μl antibody solution followed by 30 minutes incubation at 4° C. 100 μl FACS buffer was added before centrifugation and cells were washed twice with 200 μl FACS buffer. Samples were acquired subsequently on a BD LSRFortessa™ Cell Analyzer together with the BD Quantibrite™ Beads. Calculation and analysis was performed according to the manufacturer's protocol. Data was analyzed using FlowJo v10.8.1 for PC (FlowJo LLC), Microsoft Excel (Microsoft Office Standard 2016) and TIBCO SpotFire v10.10.4 (TIBCO Software Inc).

Ex Vivo Analysis of Human T Cells from Treated Humanized Mice

In one study, NSG-huNSG mice were delivered from Charles River and transferred in-house with human stem cells according to internal protocols. The humanized mice were injected i.v. with complimentary VH/VL pairs of SPLIT molecules when tumors reached a size of approximately 360 mm³. The VH and VL molecule administrations were separated by ˜20 minutes, at a dose of 1.53-1.71 mg/kg of SPLIT molecule in 100 μl of vehicle buffer (20 mM histidine, 140 mM NaCl, pH 6.0), for a total of 200 μl injected. As negative control, 200 μl of vehicle buffer was injected. In another experiment, CD34-engrafted NSG mice were delivered from Jackson Laboratories injected as described above with complimentary VH/VL pairs of SPLIT molecules (2.5-2.8 mg/kg in 100 μl per molecule).

In both studies, blood was harvested from n=3-4 mice 24 h after therapy administration and collected in heparin tubes. Erythrocytes lysis on blood samples was performed and single cells were stained for human CD45, CD8a, CD25, CD69, CD4, and PE-labeled anti-human IgG (for the detection of treatment molecules that were bound to CD4+ and CD8+ T cells). Samples were acquired on a BD LSRFortessa cell analyzer.

### 3.2 Results

As shown in FIG. 2, the molecule pairs, without complementation, composed of SPLIT V9 (A) and SPLIT P035.093 (B) assembled and bound to CD3+ Jurkat T cells in absence of target cells, while the CD3 binders 40G5c (C) and C22 (D) did not. Importantly, complementation of the split P035.093 (A) and split V9 (B) prevented the assembly on T cells in absence of target tumor cells.

As shown in FIG. 3, treatment with SPLIT pairs (HER2+HER2) and the positive control (non-SPLIT HER2×CD3 T-cell bispecific antibody (with bivalent HER2 binding and monovalent CD3 binding)) led to potent T cell mediated killing of SKOV-3 tumor cells after 48 hours of co-incubation, while the single prodrugs showed no activity. Although SPLIT complementation reduced potency compared to non-complemented pairs, this result shows that the tested SPLIT pairs, including the complemented ones, are able to induce T cell mediated tumor cell killing when both molecules bind to the target cells and assemble an active CD3 binder. It also shows that the prodrugs remain inactive in absence of their cognate partner.

The SPLIT pairs with the non-complemented CD3 binder P035.093 shows in FIG. 4 (A) that T cell mediated tumor cell killing occurs with the productive pair, but also with a pair of HER2 and an irrelevant binder. This result shows that the assembly of an active complex can occur with the non-complemented P035.093 although only one of the two molecules can bind to the tumor cell. This undesirable assembly can be prevented by complementation, as shown in FIG. 4 (B). Indeed, the HER2+HER2 complemented pair induces T cell mediated tumor cell killing, while the complemented HER2+irrelevant binder pair does not. These results show the importance of the complementation for P035.093, to restrict activity solely on target cells bound by both molecules, although this comes at the cost of slightly diminished potency.

The SPLIT pair with the non-complemented CD3 binder 40G5c shows in FIG. 5 (A) that T cell mediated tumor cell killing occurs with the productive pair (HER2+HER2), but also with a pair of HER2 and an irrelevant binder although only to a limited extent at high concentration. This result suggests that the assembly of an active complex can occur with the non-complemented 40G5c pair when both molecules can bind to the tumor cell, while the assembly of a functional complex occurs to a much lower extent if only one molecule can bind to target cells. A similar result can be observed in FIG. 5 (B) for the CD3 binder C22. Altogether, the data in FIG. 4 and FIG. 5 show that assembly of a functional complex can differ depending on the VH and VL modules and if complementation is used or not. This provides engineering opportunities to modulate the force driving the assembly of the complex.

As shown in FIG. 6 (A), the SPLIT pair with the complemented CD3 binder P035.093 confirms T cell mediated target cell killing activity when both molecules can bind to the target cells, while no killing is observed when only one molecule can bind. The assembly of an active complex does not occur on target cells with low antigen density as shown in FIG. 6 (B, C). In fact, this adds an additional requirement for the productive assembly of an active complex. Not only both molecules need to bind the target cells, but also the antigen density needs to be sufficient to trigger T cell activation and killing of target cells. The density threshold requirement may provide additional safety benefits by limiting activity on cells expressing low levels of both targets, since relevant targets for T cell redirection in oncology are frequently observed on healthy tissues, albeit at lower levels than on tumor cells.

As shown in FIG. 7 (B), non-complemented SPLIT molecules may or may not be active on low target expressing cells, depending on their CD3 binder, despite being active on high target expressing cells (FIG. 7 (A)).

The ex vivo analysis of human T cells from humanized mice sampled 24 h after treatment with SPLIT pairs shows in FIG. 8 that the non-complemented pair using P035.093 as CD3 binder can assemble in absence of binding to the target pair and bind to CD3 on CD4+ (A) and CD8+ T cells (B) circulating in the blood. Complementation of the P035.093 CD3 module prevents the assembly of the pair on T cells in circulation.

The ex vivo analysis of human T cells from humanized mice sampled 24 h after treatment with SPLIT pairs shows in FIG. 9 that the non-complemented pair using V9 as CD3 binder can assemble in absence of binding to the target pair and bind to CD3 on CD4+ (A) and CD8+ T cells (B) circulating in the blood. Complementation of the V9 CD3 module strongly reduced the assembly of the pair on T cells in circulation. Altogether, FIG. 8 and FIG. 9 show that complementation of the SPLIT CD3 improves the behavior of SPLIT compounds in circulation to limit assembly on T cells when the molecules are not bound to their target pair.

The ex vivo analysis of human T cells from humanized mice sampled 24 h after treatment with SPLIT pairs shows in FIG. 10 that other CD3 binders (e.g. 40G5c and C22) do not require complementation to prevent binding to CD3 on CD4+ (A) and CD8+ T cells (B) circulating in the blood.

### * * *

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A pair of binding molecules, comprising
(a) a first binding molecule comprising (i) a first antigen binding domain capable of binding to a target antigen, (ii) a first part of an effector domain, and (iii) a first complementing domain capable of association with the first part of the effector domain; and
(b) a second binding molecule comprising (i) a second antigen binding domain capable of binding to a target antigen, (ii) a second part of an effector domain, and (iii) a second complementing domain capable of association with the second part of the effector domain;
wherein the first and the second part of the effector domain are capable of associating with each other to form a functional effector domain if the first and the second antigen binding domain bind to their target antigens on the surface of a cell,
wherein the first and the second complementing domain are associated with the first and the second part of the effector domain, respectively, while the first and the second part of the effector domain are not associated with each other; and
wherein the first and the second complementing domain are not capable of forming a functional effector domain with a part of the effector domain.

2. (canceled)

3. The pair of binding molecules of claim 1, wherein the effector domain is an anti-CD3 antigen binding domain.

4.-6. (canceled)

7. The pair of binding molecules of claim 1, wherein the first part of the effector domain comprises a heavy chain variable region (VH) and the second part of the effector domain comprises a light chain variable region (VL).

8. The pair of binding molecules of claim 7, wherein
(a) the VH comprises a heavy chain complementarity determining region (HCDR) 1 of SEQ ID NO: 15, the HCDR 2 of SEQ ID NO: 16 and the HCDR 3 of SEQ ID NO: 17, and the VL comprises a light chain complementarity determining region (LCDR) 1 of SEQ ID NO: 19, a LCDR 2 of SEQ ID NO: 20 and a LCDR 3 of SEQ ID NO: 21; or
(b) the VH comprises a heavy chain complementarity determining region (HCDR) 1 of SEQ ID NO: 23, the HCDR 2 of SEQ ID NO: 24 and the HCDR 3 of SEQ ID NO: 25, and the VL comprises a light chain complementarity determining region (LCDR) 1 of SEQ ID NO: 27, a LCDR 2 of SEQ ID NO: 28 and a LCDR 3 of SEQ ID NO: 29; or
(c) the VH comprises a heavy chain complementarity determining region (HCDR) 1 of SEQ ID NO: 31, the HCDR 2 of SEQ ID NO: 32 and the HCDR 3 of SEQ ID NO: 33, and the VL comprises a light chain complementarity determining region (LCDR) 1 of SEQ ID NO: 35, a LCDR 2 of SEQ ID NO: 36 and a LCDR 3 of SEQ ID NO: 37; or
(d) the VH comprises a heavy chain complementarity determining region (HCDR) 1 of SEQ ID NO: 23, the HCDR 2 of SEQ ID NO: 24 and the HCDR 3 of SEQ ID NO: 60, and the VL comprises a light chain complementarity determining region (LCDR) 1 of SEQ ID NO: 27, a LCDR 2 of SEQ ID NO: 28 and a LCDR 3 of SEQ ID NO: 29.

9. The pair of binding molecules of claim 8, wherein
(a) the VH comprises an amino acid sequence that is at least about 95%, 96%, 97%, 98%, 99% or 100% identical to the amino acid sequence of SEQ ID NO: 18, and/or the VL comprises an amino acid sequence that is at least about 95%, 96%, 97%, 98%, 99% or 100% identical to the amino acid sequence of SEQ ID NO: 22; or
(b) the VH comprises an amino acid sequence that is at least about 95%, 96%, 97%, 98%, 99% or 100% identical to the amino acid sequence of SEQ ID NO: 26, and/or the VL comprises an amino acid sequence that is at least about 95%, 96%, 97%, 98%, 99% or 100% identical to the amino acid sequence of SEQ ID NO: 30; or
(c) the VH comprises an amino acid sequence that is at least about 95%, 96%, 97%, 98%, 99% or 100% identical to the amino acid sequence of SEQ ID NO: 34, and/or the VL comprises an amino acid sequence that is at least about 95%, 96%, 97%, 98%, 99% or 100% identical to the amino acid sequence of SEQ ID NO: 38; or
(d) the VH comprises an amino acid sequence that is at least about 95%, 96%, 97%, 98%, 99% or 100% identical to the amino acid sequence of SEQ ID NO: 61, and/or the VL comprises an amino acid sequence that is at least about 95%, 96%, 97%, 98%, 99% or 100% identical to the amino acid sequence of SEQ ID NO: 30.

10.-16. (canceled)

17. The pair of binding molecules of claim 1, wherein the first complementing domain comprises a VL and the second complementing domain comprises a VH, wherein the VH and the VL are non-antigen binding.

18. (canceled)

19. The pair of binding molecules of claim 17, wherein the VH comprises a heavy chain complementarity determining region (HCDR) 1 of SEQ ID NO: 47, the HCDR 2 of SEQ ID NO: 48 and the HCDR 3 of SEQ ID NO: 49, and the VL comprises a light chain complementarity determining region (LCDR) 1 of SEQ ID NO: 51, a LCDR 2 of SEQ ID NO: 52 and a LCDR 3 of SEQ ID NO: 53.

20. (canceled)

21. The pair of binding molecules of claim 1, wherein the first part of the effector domain comprises a first VH and the first complementing domain comprises a first VL, and the second part of the effector domain comprises a second VL and the second complementing domain comprises a second VH.

22. The pair of binding molecules of claim 21, wherein each of the second VH and the first VL and/or each of the first VH and the second VL comprise an amino acid substitution wherein an amino acid residue is substituted for a charged replacement amino acid residue, wherein (i) the replacement amino acid residues in the VH and the VL are of opposite charge, or (ii) the replacement amino acid residues in the VH and the VL are of the same charge.

23. The pair of binding molecules of claim 1, wherein the first binding molecule comprises a third antigen binding domain capable of binding to a target antigen, and/or the second binding molecule comprises a fourth antigen binding domain capable of binding to a target antigen.

24.-34. (canceled)

35. The pair of binding molecules of claim 1, wherein the first binding molecule comprises a first Fc domain composed of a first and a second subunit, and/or the second binding molecule comprises a second Fc domain composed of a first and a second subunit.

36. (canceled)

37. The pair of binding molecules of claim 35, wherein
the Fc domain is a human Fc domain;
the Fc domain is a human IgG Fc domain;
the Fc domain comprises a modification promoting the association of the first and the second subunit of the Fc domain; and/or
the Fc domain comprises one or more amino acid substitution that reduces binding to an Fc receptor and/or effector function.

38.-40. (canceled)

41. The pair of binding molecule of claim 1, wherein
the first binding molecule comprises (i) a first and optionally a third antigen binding domain, (ii) an Fc domain composed of a first and a second subunit, (iii) a first part of an effector domain, and (iv) a first complementing domain,
wherein
(a) the first and the third (where present) antigen binding domain is fused at its C-terminus to the N-terminus of one of the subunits of the Fc domain,
(b) the first part of the effector domain is fused at its N-terminus to the C-terminus of one of the subunits of the Fc domain,
(c) the first complementing domain is fused at its N-terminus to the C-terminus of the first part of the effector domain; and
the second binding molecule comprises (i) a second and optionally a fourth antigen binding domain, (ii) an Fc domain composed of a first and a second subunit, (iii) a second part of an effector domain, and (iv) a second complementing domain,
wherein
(a) the second and the fourth (where present) antigen binding domain is fused at its C-terminus to the N-terminus of one of the subunits of the Fc domain,
(b) the second part of the effector domain is fused at its N-terminus to the C-terminus of one of the subunits of the Fc domain,
(c) the second complementing domain is fused at its N-terminus to the C-terminus of the second part of the effector domain.

42. The pair of binding molecules of claim 1, wherein the first part of the effector domain and the first complementing domain, and/or the second part of the effector domain and the second complementing domain are fused via a peptide linker.

43. (canceled)

44. A binding molecule that forms part of the pair of binding molecules of claim 1.

45. An isolated polynucleotide encoding the pair of binding molecules of claim 1.

46. A host cell comprising the isolated polynucleotide of claim 45.

47. A method of producing a pair of binding molecules, comprising the steps of (a) culturing the host cell of claim 46 under conditions suitable for the expression of the pair of binding molecules and optionally (b) recovering the pair of binding molecules.

48. A pharmaceutical composition comprising the pair of binding molecules of claim 1 and a pharmaceutically acceptable carrier.

49.-54. (canceled)

55. A method of treating cancer in an individual, comprising administering to said individual an effective amount of the pair of binding molecules of claim 1.

56. (canceled)

57. A kit for the treatment of cancer, comprising (i) a container comprising a pharmaceutical composition, wherein the pharmaceutical composition comprises the pair of binding molecules of claim 1 and a pharmaceutically acceptable carrier, and (ii) a label or package insert containing instructions for using the pharmaceutical composition in the treatment of the disease.

58. A kit for the treatment of cancer, comprising (i) a first container comprising a first pharmaceutical composition, wherein the first pharmaceutical composition comprises the first binding molecule of the pair of binding molecules of claim 1 and a pharmaceutically acceptable carrier, (ii) a second container comprising a second pharmaceutical composition, wherein the second pharmaceutical composition comprises the second binding molecule of the pair of binding molecules of claim 1 and a pharmaceutically acceptable carrier, and optionally (iii) a label or package insert containing instructions for using the first and the second pharmaceutical composition in combination in the treatment of the disease.

59. (canceled)

60. A method for forming a functional effector domain, comprising contacting the pair of binding molecules of claim 1 with a cell expressing the target antigens of the first and the second antigen binding domains, under conditions allowing binding of the first and the second antigen binding domain to their target antigens on the surface of the cell.

61. (canceled)
